(19) United States
(12) Patent Application Publication
(10) Pub. No.: US 2023/0130579 A1
(43) Pub. Date: Apr. 27, 2023

(54) REGULATION OF POST-ISCHEMIC INFLAMMATORY RESPONSE: A NOVEL FUNCTION OF TYROSINE PHOSPHATASE STEP

(71) Applicant: UNM Rainforest Innovations, Albuquerque, NM (US)

(72) Inventors: Surojit Paul, Albuquerque, NM (US); Ranjana Poddar, Albuquerque, NM (US)

(21) Appl. No.: 17/759,864

(22) PCT Filed: Feb. 1, 2021
(86) PCT No.: PCT/US21/16048

§ 371 Date: Aug. 1, 2022

Related U.S. Application Data
(60) Provisional application No. 62/968,270, filed on Jan. 31, 2020.

(51) Int. Cl. A61K 38/46; C12N 9/16; A61P 29/00

(52) U.S. Cl. CPC A61K 38/465; C12N 9/16; C12Y 301/03048; A61P 29/00

(57) ABSTRACT

A method for the prevention, treatment, or amelioration of a medical disease or condition associated with inflammation caused by glutamate excitotoxicity comprising administering to a patient a peptide that binds to or interferes with the P38 MAPK-COX2-PGE₂ and ERK MAPK-CX3CL1-sCX3CL1 pathways.

### CROSS-REFERENCE TO RELATED APPLICATIONS

The following application claims benefit of U.S. Provisional Application No. 62/968,270, filed Jan. 31, 2020, which is hereby incorporated by reference in its entirety.

### STATEMENT REGARDING GOVERNMENT SPONSORED RESEARCH

This invention was made with Government support under Grant No. RO1 NS059962 (PI: Paul S) awarded by the National Institute of Neurological Disorders and Stroke (NIH/NINDS). The U.S. Government has certain rights in this invention.

### BACKGROUND

Glutamate is the major excitatory neurotransmitter in the brain and is involved virtually in all activities of the central nervous system. Under physiological conditions extracellular glutamate concentration in the brain is maintained in the low micromolar range by the excitatory amino acid transporters, as increase in extracellular glutamate concentration may lead to excessive activation of glutamate receptors in the nerve cells (Lewernz and Maher, 2015; Olney, 1986). Such excessive activation of glutamate receptors, also known as excitotoxicity, has been shown to play a pivotal role in brain injury in a range of neurological disorders that include acute insults like ischemic stroke and traumatic brain injury as well as chronic neurodegenerative diseases that includes Huntington's disease, Alzheimer's disease, Parkinson's disease and amyotrophic lateral sclerosis (Lewernz and Maher, 2015; Dong et al., 2009; Olloquequi et al., 2018; Lai et al., 2014). These findings suggest that excitotoxicity is a common pathogenic pathway in neurodegenerative disorders with distinctly different genetic etiology.

Glutamate can bind to the ionotropic glutamate receptor subtypes α-amino-3-hydroxy-5-methyl-4-isoxazolepropionic acid (AMPA), kainate, and N-methyl-D-aspartic acid (NMDA), and the metabotropic family of glutamate receptors (mGluRs) (Lodge 2009; Spooren et al., 2010). However, the excitotoxic effects of glutamate are mediated primarily through the NMDA subtype of ionotropic glutamate receptors, which has the highest affinity for glutamate (Waxman and Lynch 2005). Excessive stimulation of neuronal NMDARs during an excitotoxic insult leads to intracellular Ca²⁺ overload resulting in the activation of deleterious cascade of events that eventually leads to neurotoxicity and brain damage (Lynch & Guttman, 2002). Emerging studies indicate that excitotoxicity may also trigger inflammatory response in the brain (Olloquequi et al., 2018; Wang and Michaelis, 2010; Dong et al., 2009; Dantzer and Walker 2014; Vivani et al., 2014; Haroon et al., 2016). However, the underlying mechanisms through which excessive activation of glutamate receptors could enhance inflammatory response in the brain is not well understood.

### SUMMARY

According to various embodiments, the present disclosure provides methods and apparatus for the regulation of inflammatory response in neurons and in the brain following excitotoxic insult. The scientific findings herein show that the hallmarks of the increased inflammatory response following excitotoxicity includes sustained activation of two mitogen activated protein kinases (MAPKs): p38 MAPK and the extracellular regulated kinase (ERK) MAPK in neurons resulting in the increased release of inflammatory mediators. According to some embodiments, the methods and apparatus exploit the role of tyrosine phosphatase in the brain in order to regulate the inflammatory response. According to a specific embodiment, these methods and apparatus involve the utilization of a STEP-derived peptide mimetic as a control point in the regulation of pro-inflammatory response in neurons following the insult. Specifically, in the ex vivo studies in neuron cultures from STEP-deficient mice the enhanced neuronal release of the pro-inflammatory prostanoid prostaglandin E2 (PGE₂) and the neuron-specific chemokine, CX3CL1 following an excitotoxic insult, as well as the attenuation of such inflammatory response with restoration of STEP signaling using a STEP peptide mimetic provides therapeutic opportunity. In additional embodiments the methods and apparatus exploit ischemic stroke as an animal model of excitotoxicity to demonstrate increased inflammatory response in STEP deficient mice following cerebral ischemia. Similar to the observation in the ex vivo studies, the enhanced inflammatory response in STEP KO mice following stroke involves increased PGE₂ and CX3CL1 release, resulting in microglial activation, blood-brain barrier disruption and peripheral immune cell infiltration. Restoration of STEP signaling with intravenous administration of the STEP peptide mimetic attenuates this inflammatory response. The increased inflammatory response in both ex vivo and in vivo studies suggest that the STEP enzyme plays a far more important role in brain function than just regulating ischemia-induced brain injury. The findings also highlight the therapeutic potential of the STEP-peptide mimetic in a wide spectrum of neurological disorders where excitotoxicity contributes to brain damage through activation of inflammatory pathways.

### BRIEF DESCRIPTION OF THE FIGURES

FIG. 1A is a schematic representation of the signaling cascade of p38 MAPK in wildtype mice neurons following an excitotoxic insult.

FIG. 1B is a schematic representation of the signaling cascade of p38 MAPK involved in increased PGE₂ release in the neurons of STEP KO mice following an excitotoxic insult.

FIG. 2A is a schematic representation of the signaling cascade of ERK MAPK in wildtype mice neurons following an excitotoxic insult.

FIG. 2B is a schematic representation of the signaling cascade of ERK MAPK involved in increased PGE₂ release from STEP KO mice neurons following an excitotoxic insult.

FIG. 3A shows glutamate induced phosphorylation of p38 MAPK in neurons of wild type mice.

FIG. 3B shows glutamate induced phosphorylation of p38 MAPK in neurons of STEP knockout mice.

FIG. 3C is the quantified results from FIGS. 3A and 3B.

FIG. 4A shows the temporal profile of neuronal p38 MAPK phosphorylation in the post-glutamate time period in wild type mice.

FIG. 4B shows the temporal profile of neuronal p38 MAPK phosphorylation in the post-glutamate time period in STEP knockout mice.

FIG. 4C is the quantified results from FIGS. 4A and 4B.

FIG. 5A shows the effect an excitotoxic insult on cPLA2 activity following deletion of endogenous STEP.

FIG. 5B shows that application of either MK801 or SB 203580 during insult significantly reduced cPLA2 activity observed 2 h after insult.

FIG. 6A shows the evaluation of the expression of COX2 in wildtype mice.

FIG. 6B shows the evaluation of the expression of COX2 in STEP KO mice.

FIG. 6C is quantification of the results of the experiments in FIGS. 6A and 6B.

FIG. 6D is a pharmacological study showing that inhibition of NMDAR with MK801 attenuated glutamate-induced increase in COX2 levels in the STEP KO mice neurons.

FIG. 6E is a pharmacological study showing that inhibition of p38 MAPK activation with SB 203580 attenuated glutamate-induced increase in COX2 level in the STEP KO mice neurons.

FIG. 7A demonstrates the role of NMDA receptor, p38 MAPK and COX-2 in glutamate induced PGE₂ release from in neurons.

FIG. 7B shows that glutamate-induced PGE₂ release from STEP KO mice neurons is significantly reduced following inhibition of NMDAR, p38 MAPK and COX2.

FIG. 8A shows the effect of glutamate treatment on NF□B activation in neurons from WT mice.

FIG. 8B shows the effect of glutamate treatment on NF□B activation in neurons from STEP KO mice.

FIG. 8C is quantitation of the results in FIGS. 8A and 8B.

FIG. 9A shows the role of NF□B activation in the absence or presence of MK801.

FIG. 9B shows the role of NF□B activation in the absence or presence of SB203580.

FIG. 9C shows the role of NF□B activation in glutamate induced COX2 expression in the absence or presence of Bengamide B.

FIG. 9D shows the role of NF□B activation in glutamate induced PGE₂ expression in the absence or presence of Bengamide B.

FIG. 10 is a schematic representation of an exemplary TAT-STEP-myc peptide generated from STEP₆₁.

FIG. 11A shows the effect of the STEP peptide mimetic (TAT-STEP-myc) shown in FIG. 10 on glutamate-induced increase in p38 MAPK phosphorylation.

FIG. 11B shows the effect of the STEP peptide mimetic (TAT-STEP-myc) shown in FIG. 10 on glutamate-induced increase in COX2 expression.

FIG. 11C shows the effect of the STEP peptide mimetic (TAT-STEP-myc) shown in FIG. 10 on glutamate-induced increase in PGE₂ release.

FIG. 12A shows p38 MAPK phosphorylation in striatum from WT mice subjected to sham surgery.

FIG. 12B shows p38 MAPK phosphorylation in striatum from STEP KO mice subjected to sham surgery.

FIG. 12C shows p38 MAPK phosphorylation in cortex from WT mice subjected to sham surgery.

FIG. 12D shows p38 MAPK phosphorylation in cortex from STEP KO mice subjected to sham surgery.

FIG. 12E shows p38 MAPK phosphorylation in striatum from WT mice subjected to sham surgery followed by reperfusion.

FIG. 12F shows p38 MAPK phosphorylation in striatum from STEP KO mice subjected to sham surgery followed by reperfusion.

FIG. 12G shows p38 MAPK phosphorylation in cortex from WT mice subjected to sham surgery followed by reperfusion.

FIG. 12H shows p38 MAPK phosphorylation in cortex from STEP KO mice subjected to sham surgery followed by reperfusion.

FIG. 12I shows p38 MAPK phosphorylation in striatum from WT and STEP KO mice subjected to MCAO.

FIG. 12J shows p38 MAPK phosphorylation in cortex from WT and STEP KO mice subjected to MCAO.

FIG. 12K shows immunohistochemical staining of coronal brain sections with anti-phospho-p38 MAPK and NeuN antibodies.

FIG. 13A shows COX2 protein levels in striatum from WT mice subjected to sham surgery.

FIG. 13B shows COX2 protein levels in striatum from STEP KO mice subjected to sham surgery.

FIG. 13C shows COX2 protein levels in cortex from WT mice subjected to sham surgery.

FIG. 13D shows COX2 protein levels in cortex from STEP KO mice subjected to sham surgery.

FIG. 13E shows COX2 protein levels in striatum from WT mice subjected to sham surgery followed by reperfusion.

FIG. 13F shows COX2 protein levels in striatum from STEP KO mice subjected to sham surgery followed by reperfusion.

FIG. 13G shows COX2 protein levels in cortex from WT mice subjected to sham surgery followed by reperfusion.

FIG. 13H shows COX2 protein levels in cortex from STEP KO mice subjected to sham surgery followed by reperfusion.

FIG. 13I shows COX2 protein levels in striatum from WT and STEP KO mice subjected to MCAO.

FIG. 13J shows COX2 protein levels in cortex from WT and STEP KO mice subjected to MCAO.

FIG. 13K shows immunohistochemical staining from WT and STEP KO mice with anti-COX2 and NeuN antibodies.

FIG. 13L shows PGE₂ levels from striatum obtained from WT and STEP KO mice subjected to sham surgery followed by reperfusion.

FIG. 13M shows PGE₂ levels from cortex obtained from WT and STEP KO mice subjected to sham surgery followed by reperfusion.

FIG. 14A shows post-ischemic p38 MAPK phosphorylation in striatum from WT and STEP KO mice.

FIG. 14B shows post-ischemic p38 MAPK phosphorylation in cortex from WT and STEP KO mice.

FIG. 14C shows post-ischemic COX2 expression in striatum from WT and STEP KO mice.

FIG. 14D shows post-ischemic COX2 expression in cortex from WT and STEP KO mice.

FIG. 14E shows post-ischemic PGE₂ release in striatum from WT and STEP KO mice.

FIG. 14F shows post-ischemic PGE₂ release in cortex from WT and STEP KO mice.

FIG. 15A presents immunohistochemical staining with anti-Iba-1 antibody of coronal brain sections through cortex and striatum of WT mice.

FIG. 15B presents immunohistochemical staining with anti-Iba-1 antibody of coronal brain sections through cortex and striatum of STEP KO mice.

FIG. 15C presents immunohistochemical staining with anti-Iba-1 antibody of coronal brain sections through cortex and striatum of STEP KO mice treated with TAT.

FIG. 15D quantifies the data shown in FIGS. 15A-C.

FIG. 16A shows a FRET peptide immunoassay in striatum of WT and STEP KO mice post-MCAO reperfusion.

FIG. 16B shows a FRET peptide immunoassay in cortex of WT and STEP KO mice post-MCAO reperfusion.

FIG. 16C shows immunoblot analysis with anti-ZO-1 antibody of striatal lysates obtained from WT and WT and STEP KO mice post-MCAO reperfusion.

FIG. 16D shows immunoblot analysis with anti-ZO-1 antibody of cortical lysates obtained from WT and WT and STEP KO mice post-MCAO reperfusion.

FIG. 16E shows immunoblot analysis with anti-occludin antibody of striatal lysates obtained from WT and WT and STEP KO mice post-MCAO reperfusion.

FIG. 16F shows immunoblot analysis with anti-occludin antibody of cortical lysates obtained from WT and WT and STEP KO mice post-MCAO reperfusion.

FIG. 16G shows IgG concentration in the striatal lysates obtained from ipsilateral hemisphere of WT and STEP KO mice after post-MCAO reperfusion was measured by enzyme immunoassay.

FIG. 16H shows IgG concentration in the cortical lysates obtained from ipsilateral hemisphere of WT and STEP KO mice after post-MCAO reperfusion was measured by enzyme immunoassay.

FIG. 17A shows MMP-9 activation in the striatum from WT, STEP KO, and TAT-STEP treated STEP KO mice.

FIG. 17B shows MMP-9 activation in the cortex from WT, STEP KO, and TAT-STEP treated STEP KO mice.

FIG. 17C shows IgG concentration of striatum lysates from WT, STEP KO, and TAT-STEP treated STEP KO mice.

FIG. 17D shows IgG concentration of cortex lysates from WT, STEP KO, and TAT-STEP treated STEP KO mice.

FIG. 18A shows ERK MAPK phosphorylation in neuron cultures from WT mice.

FIG. 18B shows ERK MAPK phosphorylation in neuron cultures from STEP KO mice.

FIG. 18C shows CX3CL1 levels in neuron cultures from WT and STEP KO mice.

FIG. 18D shows ERK levels in cell lysates of STEP KO mice in the absence or presence of TAT STEP.

FIG. 18E shows CX3CL1 levels in the absence or presence of TAT STEP.

FIG. 19A shows the ERK levels as measured by ELISA of the striatum and cortex of WT and STEP K mice.

FIG. 19B shows the CX3CL1 levels as measured by ELISA of the striatum and cortex of WT and STEP K mice.

FIG. 19C shows quantitation of the data shown in FIG. 19B.

FIG. 19D shows ERK MAPK phosphorylation evaluated by immunoblot analysis of tissue extracts from ipsilateral striatum and cortex and re-probed with anti-ERK MAPK.

FIG. 19E shows CX3CL1 protein levels evaluated by immunoblot analysis of tissue extracts from ipsilateral striatum and cortex and re-probed with anti-β-tubulin antibody.

FIG. 20A shows the results of neurological dysfunction tests using a 5-point neurological severity score.

FIG. 20B shows balance and coordinated alteration of fore- and hind-paw as evaluated using the rotarod.

FIG. 20C shows gait and balance evaluated using a beam balance task.

FIG. 20D shows representative photomicrographs of coronal brain sections obtained from WT, STEP KO and STEP KO mice treated with the STEP peptide mimetic demonstrating effect of the STEP-peptide mimetic on the progression of ischemic brain damage.

FIG. 21 is a schematic representation of the proposed inflammatory pathways that are involved in brain injury in the absence of STEP.

### DETAILED DESCRIPTION

According to an embodiment the present disclosure provides methods and apparatus for the regulation of inflammatory response triggered by excitotoxicity. In general, the present disclosure provides for the delivery of a synthetic STEP peptide mimic to prevent, ameliorate, or treat medical conditions associated with inflammatory responses triggered by excitotoxicity. The present disclosure presents evidence evaluating the functional significance of excitatory NMDAR stimulation in neuronal cultures obtained from wild-type (WT) and STEP knock out (KO) mice. The findings presented herein indicate that in the neurons from STEP KO mice an excitatory insult with glutamate triggers an inflammatory response through the sustained increase in phosphorylation and subsequent activation of p38 MAPK and ERK MAPK, the two substrates of STEP. p38 MAPK activation leads to increased activation of cytosolic phospholipase-2 (cPLA2) and nuclear factor B (NF-κB), which are key regulators of p38 MAK-dependent expression of cyclooxygenase-2 (COX2), a signaling pathway involved in PGE₂ biosynthesis (Hwang et al., 1997; Ridley et al., 1998; Paul et al., 1999). The findings further show that the excitatory insult significantly increased PGE₂ release from STEP KO mice neurons. However, it had no effect on PGE₂ release from WT mice neurons. Using the STEP peptide-mimetic the study further establishes STEP as a key regulator of neuronal p38 MAPK activation, COX2 expression, and PGE₂ release. Finally, the study shows that the delivery of a synthetic STEP mimetic peptide significantly reduces the inflammatory response.

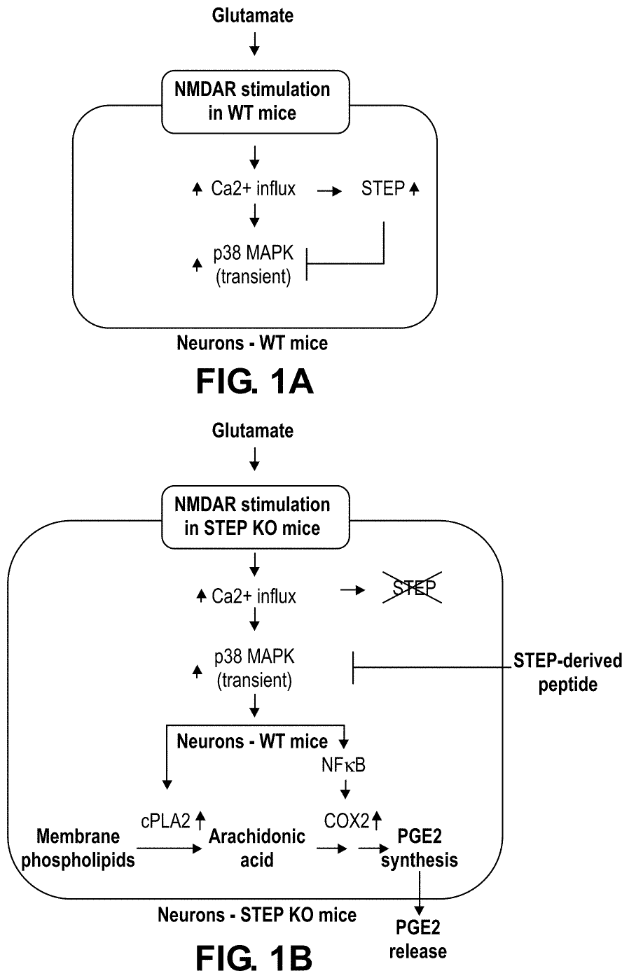

The p38 MAPK signaling cascade following an excitotoxic insult in wild type and STEP knockout mice are shown respectively in FIGS. 1A and 1B. It can be seen that an excitotoxic insult with glutamate in WT mice neurons leads to transient increase in p38 MAPK activation that fails to induce increase in neuronal PGE₂ synthesis and release. However, in neurons from STEP KO mice, glutamate-mediated NMDAR stimulation induces sustained activation of p38 MAPK, which leads to concomitant increase in cPLA2 and NFκB activity resulting in COX2-mediated PGE₂ synthesis and release from neurons. The findings reveal a novel role of STEP in regulating neuroinflammation.

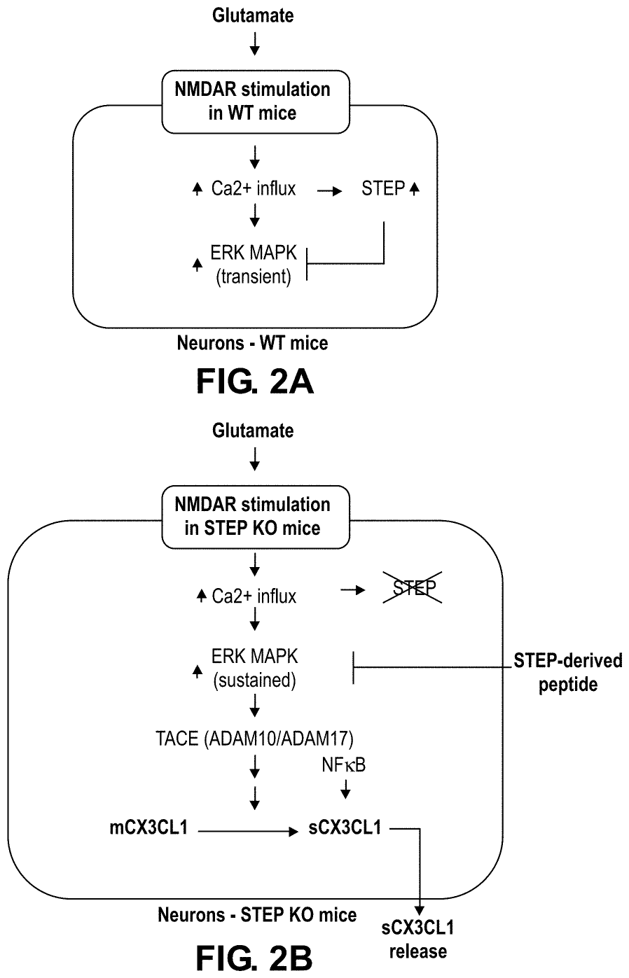

FIGS. 2A and 2B show the ERK MAPK signaling cascade in wild type and STEP KO mice, respectively. As shown, ERK MAPK activation leads to an increase in the release of the chemokine domain of CX3CL1, a member of the chemokine family that is expressed predominantly in neurons in the brain and is known to promote microglial activation during inflammatory episodes (Sheridan et al., 2013; Nishiyori et al., 1998; Rostene et al., 2007; Harrison et al., 1998). CX3CL1 is a membrane anchored protein and ERK MAPK mediated activation of the disintegrin-like metalloproteinase, ADAM10 is known to cleave CX3CL1 and release the soluble chemokine domain of CX3CL1 (Wan et al., 2012; Hundhausen et al., 2003, 2007). Restoration of STEP signaling with the STEP peptide mimetic attenuates ERK MAPK activation and soluble CX3CL1 release.

These findings reveal a novel role of STEP in regulating neuroinflammation. Accordingly, the present disclosure uses this knowledge to develop a novel treatment regime for neuroinflammation.

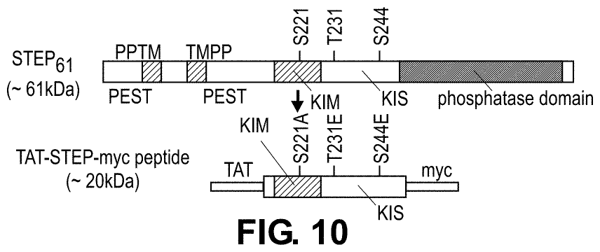

While the present disclosure provides discussion and experimental results directed primarily towards the STEP-derived peptide shown in FIG. 10, it will be understood that numerous variations of said peptide could also be used, including sequence additions, deletions and/or modifications to include a variety of functional or non-functional modifications and/or elements including, but not limited to, markers, signaling, etc.

Accordingly, the present disclosure provides a synthetic peptide that constitutively binds p38 MAPK and/or ERK MAPK and the use thereof in the treatment, abatement, or prevention of conditions associated with the neuronal p38 MAPK-COX2-PGE₂ signaling cascade, ERK MAPK-CX3CL1 signaling cascade, microglial activation, MMP-9 activity and/or IgG extravasation including, but not necessarily limited to, inflammatory responses associated with stroke, head trauma, depression, multiple sclerosis, epilepsy, Huntington's disease, Alzheimer's disease, Parkinson's disease and amyotrophic lateral sclerosis.

According to a specific embodiment, the synthetic peptide is a STEP peptide mimetic. The STEP protein mimetic may contain a number of modifications from the naturally occurring STEP peptide which enable it to be administered as a therapeutic. For the purpose of the present disclosure, unless otherwise clear from context, the term “modification” in the context of a “modified peptide” refers to alterations in the mimetic peptide when compared to its naturally occurring counterpart. For example, the peptide sequence may be designed to direct the mimetic to a specific region or target. For example, the inclusion of a Trans-Activator of Transcription of human immunodeficiency virus (TAT) nucleotide sequence in the peptide renders the mimetic peptide cell permeable. Moreover, the peptide sequence may be designed such that the mimetic peptide is both stable (i.e. less likely to degrade) and constitutively active (i.e. permanently binds its target sequence.) As specific examples, stability of the STEP peptide may be increased by modifying the KIS domain to mimic phosphorylation while modifications to the KIM domain render the peptide constitutively active.

The present disclosure envisions the delivery of a STEP peptide mimetic to a patient suffering from any number of inflammatory conditions including, for example, neurological disorders that include acute insults like ischemic stroke and traumatic brain injury as well as chronic neurodegenerative diseases that includes Huntington's disease, Alzheimer's disease, Parkinson's disease and amyotrophic lateral sclerosis. The degree and/or type of disease or disorder, among other factors, will typically determine the method and timing of delivery, which may have implications for the specific design of the mimetic. For example, an acute condition like stroke or traumatic brain injury may require a single intravenous dosage delivered as close to the time of injury as possible. On the other hand, chronic conditions like Alzheimer's or Parkinson's may require repeated dosage over time.

FIG. 10 is a schematic illustration of the STEP₆₁ protein (upper) and an exemplary STEP peptide mimetic (lower) according to an embodiment of the present disclosure. As shown, the schematic diagram of STEP₆₁ indicates the positions of the phosphatase domain, putative proteolytic sites (PEST), transmembrane domain (TM), polyproline rich regions (PP), kinase interacting motif (KIM), kinase specificity sequence (KIS) and known phosphorylation sites. The STEP derived peptide (TAT-STEP-myc) was rendered cell-permeable by fusion to the 11 amino acid protein transduction domain (TAT) of the human immunodeficiency virus-type I at the N-terminus and has a myc-tag at the C-terminus. The serine residue in the kinase interacting motif (KIM) was mutated to alanine to allow the peptide to bind constitutively with its substrates. The threonine and serine residues in the kinase specificity sequence (KIS) were mutated to glutamic acid to render the peptide resistant to degradation.

According to another embodiment, a shorter mimetic peptide, containing, for example, only the modified KIM domain (without the modified KIS domain) may be more suitable and easier to handle for repeated dosage, such as would be needed for chronic conditions including STEP-related conditions associated with aging.

According to one embodiment, the synthetic peptide of the present disclosure is a peptide that constitutively binds p38 MAPK and/or ERK MAPK. This embodiment may include a modified version of some or all the STEP KIM domain.

As stated above, the present disclosure provides for the prevention, amelioration, and/or treatment of a variety medical conditions associated with inflammatory responses triggered by excitotoxicity. According to one embodiment, the present disclosure provides for the present disclosure provides for the prevention, amelioration, and/or treatment of a variety medical conditions associated with inflammatory responses caused by aging. Aging has profound impact on the cerebrovasculature in humans that result in significant reduction in cerebral blood flow and oxygen consumption (Ainslie et al., 2008; De Vis et al., 2015; Candelario-Jalil and Paul 2020). The resulting hypoxia increases the susceptibility of the aged brain to oxidative stress with neurons and endothelial cells being the most vulnerable (Macri et al., 2010; Ostegaard et al., 2016). Intracellular alteration in redox homeostasis is thought to be the largest risk factor for age associated decline in cognitive and motor functions and development of both acute and chronic neurodegenerative disorders (Hekimi et al., 2011; Yankner et al., 2008). Such prooxidative shift, primarily due to depletion of brain glutathione level, disrupts redox-associated survival signaling pathways thereby increasing the susceptibility of the aging brain to functional and metabolic stressors (Curaris and Maher 2013; Ballatori et al., 2009; Emir et al, 2011). Consistent with this interpretation, earlier studies in rodents showed that depletion of glutathione level in the aging brain is associated with dimerization and loss of function of STEP, which is known to be involved in neuroprotection (Rajagopal et al., 2016). Additional studies in neuronal cultures and cell lines overexpressing STEP have demonstrated the ability of H₂O₂ induced oxidative stress and DEM mediated glutathione depletion in the dimerization and subsequent loss of function of STEP (Deb et al., 2011). The findings from the current study in conjunction with the previous research now imply that the loss of function of endogenous STEP in the aged brain combined with an increase in extracellular glutamate level in age-associated brain pathologies could exacerbate disease progression by enhancing inflammatory response.

The characterization of the role of the tyrosine phosphatase STEP in regulating the neuronal production of PGE₂ and soluble CX3CL1 reveals that STEP constitutes an important control point, restricting the early onset of inflammatory response following an excitotoxic insult. The distinctly different pattern of p38 MAPK and ERK MAPK signaling in neurons from WT and STEP KO mice brain could be attributed to the loss of STEP, as both p38 MAPK and ERK MAPK are substrates of STEP.

In the absence of this inhibitory signal (STEP) sustained activation of p38 MAPK is the initial trigger in glutamate-NMDAR mediated neuronal PGE₂ release. Our findings further show that the sustained activation of p38 MAPK plays a profound role in the neuronal PGE₂ synthesis. An early onset and substantial increase in the activity of cPLA2, the first enzyme involved in the synthesis of PGE₂, was observed in neurons from STEP KO mice, and to a much lesser extent in neuron from the WT mice. However, NFκB-mediated expression COX2 expression, the second enzyme involved in PGE₂ synthesis, was observed only in STEP KO mice neurons. The activation of cPLA2 and the expression of COX2 in different cell types are regulated by multiple signaling pathways, including p38 MAPK pathway, which has been found to be activated in several neurological disorders that includes ischemic stroke, Alzheimer's disease, Parkinson's disease and amyotrophic lateral sclerosis (Deb et al., 2013; Munoz and Ammit 2010; Kim and Choi, 2015; He et al., 2018). Earlier studies have reported that p38 MAPK phosphorylates cPLA2 at Ser505 to augment its activity (Lin et al., 1993; Kramer et al., 1996). More recent studies showed that p38 MAPK activation enhances the stability of COX2 mRNA resulting in increased protein levels (Lasa et al., 2000; Svensson et al., 2003). Consistent with these observations, we observed that inhibition of p38 MAPK activation attenuated both cPLA2 activation and COX2 expression in STEP KO mice neurons. In neurons from STEP KO mice, we also observed sustained and significant increase in PGE₂ release, which was attenuated by pharmacological inhibition of p38 MAPK, NFkB and COX2. Taken together these findings indicate that the consequences of p38 MAPK activation depend largely on the duration of its activation. A transient activation of p38 MAPK in WT mice neurons fails to exert substantial effect on PGE₂ synthesis. In contrast, the prolonged activation of p38 MAPK observed in STEP KO mice neurons is crucial for the synergistic activation of cPLA2 and elevated expression of COX2 and leads to robust increase in PGE₂ synthesis.

The sustained phosphorylation of p38 MAPK in the absence of STEP and the attenuation of p38 MAPK phosphorylation following restoration of STEP signaling with a cell-permeable STEP derived peptide reveal a novel mechanism of regulation of inflammatory response in neurons following an excitotoxic insult. In earlier studies, we have established that the interaction between STEP and its substrates (p38 MAPK and ERK MAPK) is regulated by the phosphorylation of a critical serine residue within the KIM domain of STEP. Dopamine/D1 receptor mediated phosphorylation of this serine residue renders STEP inactive in terms of its ability to bind to its substrate (Paul et al., 2000). Dephosphorylation of this residue following glutamate/NMDAR stimulation allows STEP to bind to it substrates and inhibit their activity (Paul et al., 2003; Poddar et al., 2010). Based on these findings a STEP-derived peptide was generated, where the serine residue in the KIM domain was mutated to allow the peptide to bind constitutively with its substrates, which includes p38 MAPK (Poddar et al., 2010; Poddar et al., 2019). In the current study, restoration of STEP signaling in STEP KO mice neurons with the application of this peptide not only reduced p38 MAPK phosphorylation but also attenuated COX2 expression and PGE₂ synthesis. Excessive and persistent release of PGE₂ in the brain has been associated with microglial activation and peripheral immune cell infiltration, the two cardinal features of neuroinflammation associated with acute and chronic neurological disorders (Graeber et al., 2011; Prinz and Priller 2017). As such, the efficacy of the STEP-peptide to attenuate PGE₂ release establishes the role of STEP as a regulator of neuroinflammatory response under excitotoxic conditions. This interpretation is further supported by recent findings in a STEP KO mice model of ischemic stroke demonstrating that in the absence of STEP increased microglial activation, blood brain barrier disruption and extravasation of immunoglobulins in the brain leads to exacerbation of ischemic brain injury (Rajagopal et al., 2021). The study further showed that restoration of the STEP signaling with post-stroke administration of the STEP-derived peptide resulted in attenuation microglial action and BBB disruption, and significant reduction in infarct size in the STEP KO mice.

The increase in phosphorylation of ERK MAPK following the excitotoxic insult in the ex vivo study or ischemic stroke in the in vivo study further suggest that STEP may play a role in regulating multiple inflammatory cascades associated with excitotoxicity. Consistent with this interpretation we also observed an increase in CX3CL1 level in both the cell cultures study in neurons (ex vivo) and in the ischemic brain (in vivo). The efficacy of the STEP peptide mimetic to attenuate both ERK MAPK phosphorylation and soluble CX3CL1 formation in the ex vivo and in vivo studies respectively further corroborates our hypothesis.

These findings highlight the role of STEP in neuroimmune communication and could lead to a paradigm shift in our understanding of neuroinflammatory disorders related to excitotoxicity. The findings also implicate the STEP-derived peptide as a potential therapeutic target to modulate inflammation in acute or chronic neurological disorders related to excitotoxicity. In summary, the study establishes the novel role of a tyrosine phosphatase in regulating multiple inflammatory response in neurons following an excitotoxic insult and might provide a promising new tool for targeting inflammatory response in CNS pathophysiology.

### Experimental

### Materials and Methods

Animals: STEP knockout mice (KO or STEP−/−) were developed on a C57BL6 background (Venkitaramani et al., 2009). Breeding pairs of mice with heterozygous deletion of the STEP gene were used to generate both WT and STEP KO mice. The mice were then maintained in our animal facility and were used for experiments when they were 12-14 weeks old (26-27 g). All procedures involving animal are in compliance with the ARRIVE guidelines and approved by the University of New Mexico, Health Sciences Center, Institutional Animal Care and Use Committee.

Cell culture and stimulation: For cell culture studies adult WT male and female mice and STEP KO male and female mice were mated to generate timed pregnant female mice. The males were removed from the females after 24 h period, which was considered gestational day 1. Pregnancy was verified by the presence of vaginal plugs and/or weight gain. Primary neuronal cultures were obtained from 15-day-old mice embryos as described previously (Deep et al., 2019). Briefly, the striatum and the adjoining cortex was dissected, the tissue dissociated mechanically and resuspended in Dulbecco's modified Eagle's medium/F-12 (1:1) containing 5% fetal calf serum. Cells (6×106 cells/dish) were plated on poly-D-lysine-coated tissue culture dishes (BD Biosciences) and grown for 12-14 days at 37° C. in a humidified atmosphere (95:5% air:CO₂ mixture). For receptor stimulation cells were treated with glutamate or NMDA for the indicated times and then processed for immunoblot analysis. Glycine concentration in the medium during glutamate treatment was 10 μM. In some experiments, DL-2-amino-5-phosphonopentanoic acid (APV, 200 μM), MK-801 (5 μM), SB203580 (5 μM), CAY10404 (100 nM) or Bengamide B (500 nM) were added 5 min 10 min before addition of glutamate. Some cultures were treated with the TAT-STEP-myc peptide (4 μM) prior glutamate treatment.

Construction and purification of the STEP-derived peptide TAT-STEP-myc: A recombinant DNA construct for TAT-STEP-myc peptide was generated using a bacterial expression vector, expressed in E. coli and purified as described previously (Paul et al., 2003). Briefly, STEP61 cDNA lacking the PTP domain and encoding only 173-279 amino acids was sub-cloned into a pTrc-His-myc-TOPO expression vector (Invitrogen). A 11 amino acid TAT peptide (trans-activator of transcription of human immunodeficiency virus) nucleotide sequence was inserted at the N-terminal of the STEP ΔPTP cDNA to render the peptide cell permeable (Poddar et al., 2010). A point mutation was introduced by site-directed mutagenesis (Pfu Turbo, Stratagene) at serine 221 within the KIM domain (S221A) to render the peptide constitutively active in terms of its ability to bind to its substrate. Point mutations were also introduced at threonine 231 (T231E) and serine 244 (S244E) in the KIS domain to mimic the phosphorylated form that helps to maintain the stability of STEP (Mukherjee et al., 2011). The modified TAT-STEP-myc peptide was expressed in E. coli and purified using BD-Talon resin (BD Biosciences, Bedford, Mass., USA). FIG. 10 is a schematic illustration of the resulting peptide.

Induction of transient focal cerebral ischemia: Middle cerebral artery occlusion (MCAO) was performed on male wild type and STEP KO mice (25-27 g) using the intraluminal method as described earlier (Deb et al., 2013; Longa et al., 1989). Briefly, animals were anesthetized by spontaneous inhalation of isoflurane (2%) in 70% nitrous oxide and 30% oxygen. Rectal temperature was maintained at 37±1° C. with an electrical heating pad both during surgery and recovery. The right common carotid artery (CCA) and the external carotid artery were exposed by a ventral midline incision in the neck region and clipped. Both the external carotid artery and pterygopalatine branch of the internal carotid artery were clipped to allow proper insertion of the occluding filament. A silicon-rubber-coated 6-0 monofilament (Doccol Corporation) was advanced through the CCA into the internal carotid artery to a length of 10-11 mm from the bifurcation to occlude the middle cerebral artery. Depending on the experiment the occluding filament was kept in place for 10-30 min or the filament was retracted after 30 min of occlusion to allow reperfusion for various durations (3 h-24 h). For reperfusion, the incision was closed under anesthesia and animals were allowed to recover in their cages. In some experiments, STEP KO mice received a single intravenous dose of TAT-STEP-myc peptide (3 nmol/g of body weight) through the femoral vein at the onset of reperfusion (Deb et al., 2013; Poddar et al., 2019). At the specified time points after occlusion or reperfusion WT and STEP KO mice were euthanized and used for biochemical studies, immunohistochemistry or Fluoro-Jade staining.

Immunoblotting: For cell culture studies neuronal cultures were washed with PBS (pH 7.4), containing sodium pyrophosphate and sodium vanadate as phosphatase inhibitors, and harvested in SDS sample buffer (Laemmli 1970). Equal protein from total cell lysates, estimated using BCA kit, was resolved by SDS-PAGE (7.5%) followed by western blotting on PVDF membranes.

For in vivo studies mice were decapitated after sham surgery and at specified time points after MCAO (10 and 30 min) and reperfusion (3, 6 and 24 h). Brain slices (2 mm thickness) were obtained using the coronal mice brain mold. Cortical and striatal tissue punches from the third rostral section of ipsilateral hemispheres were homogenized in Laemmli sample buffer, boiled at 100° C. for 10 min, centrifuged at 14,000×g (10 min) and processed for SDS-PAGE and immunoblotting studies with the following primary antibodies against proteins of interest: polyclonal anti-p38 (cat #: 9218) and rabbit monoclonal anti-phospho-p38 (TPEYP) from Cell Signaling (Cat #: 9215); polyclonal anti-ERK (Cat #:) from Santa Cruz Biotechnology; monoclonal anti-phospho-ERK (TPEYP) from Cell Signaling (Cat #:); polyclonal anti ADAM-10 from Abcam (Cat #: ab1997); polyclonal anti-CX3CL1 from Abcam (Cat #: ab25088); polyclonal anti-cyclooxygenase-2 (COX2) from Abcam (Cat #: ab15191); monoclonal anti-IκB from Cell Signaling (Cat #: 4814); monoclonal anti-β-tubulin from Sigma Aldrich (Cat #: T0198); monoclonal anti-STEP from Novus Biologicals (Cat #: NB300-202); polyclonal anti-zona occluden-1 (ZO-1) from Thermo Fisher (Cat #: 61-7300); and rabbit monoclonal anti-occludin from Abcam (Cat #: ab167161). Horseradish peroxidase conjugated goat anti-rabbit (1:1000-2000, Cat #: 7074) or goat anti-mouse (1:2000, Cat #: 7076) from Cell Signaling was used as secondary antibody.

Enzyme linked immunoassays: For measurement of cPLA2 activity, cell lysates were harvested in ice-cold Tris-buffered saline (pH 7.4) containing phosphatase inhibitor, sonicated 3 times with 5 s bursts and placed on ice (2 min) between each burst of sonication. The lysed cell suspensions were centrifuged at 10,600 g (10 min) and the supernatant was collected in another tube. Equal amounts of protein from the supernatant were processed for cPLA2 activity assay, using the cPLA2 activity assay kit (Cat #: 765021) from Cayman chemicals (Ann Arbor, Mich., USA), according to the manufacturer's protocol.

For measurement of PGE₂ and soluble CX3CL1 levels released from neurons, culture medium was collected from each experimental plate and centrifuged at 200 g for 5 min to remove cellular debris. Equal volume (100 μL) of the supernatant from each sample was used to determine PGE₂ and soluble CX3CL1 levels. PGE₂ level was assessed using the PGE₂ enzyme-linked immunosorbent assay (ELISA) kit (Cat #: K051-H1) from Arbor assays (Ann Arbor, Mich., USA), according to the manufacturer's instructions. Soluble CX3CL1 level was assessed using the ELISA kit (Cat #: MCX310) from R&D systems (Minneapolis, Minn., USA), according to the manufacturer's instructions.

To determine PGE₂ and CX3CL1 concentrations in brain lysates, WT and STEP KO mice were subjected to MCAO for 30 min followed by reperfusion for 6 h. Mice were euthanized after sham surgery and 6 h after post-MCAO reperfusion, and brains were rapidly removed on ice. Cortex and striatum were dissected out from the third rostral section of coronal brain slices (2 mm thickness) obtained from ipsilateral hemisphere. The tissues were homogenized separately in ice cold PBS in the presence of protease inhibitor cocktail (Roche), centrifuged at 14,000 rpm for 10 min. The supernatant was removed and equal amount of protein in each sample was estimated using BCA protein estimation kit (Pierce). Equal amount of protein from each sample (10 μg) was used for PGE₂ assay using the PGE₂ ELISA kit (K051-H1) from Arbor assays (Ann Arbor, Mich.), and CX3CL1 assay using the ELISA kit (MCX310) from R&D systems (Minneapolis, Minn.), according to the manufacturer's protocol.

To determine immunoglobulin G (IgG) level in the brain as a measure of blood-brain barrier (BBB) permeability WT and STEP KO mice were subjected to MCAO for 30 min followed by reperfusion for 24 h. Mice were rapidly decapitated after sham surgery and 24 h after post-MCAO reperfusion, brains were removed and cortical and striatal lysates from the ipsilateral hemisphere were obtained as described above. The tissue was homogenized separately in RIPA buffer containing 1% SDS, 1% sodium deoxycholate, 150 mM NaCl, 50 mM Tris-HCl pH 7.6, and 1% IGEPAL CA-630 (Sigma-Aldrich) at 10 μL/mg of brain tissue. HALT Protease Inhibitor Cocktail, HALT Phosphatase Inhibitor Cocktail and 0.5 M EDTA were added at 10 μL/mL of homogenization buffer immediately before use. Homogenates were spun down at 14,000×g at 4° C. for 20 min and supernatant was removed and stored at −80° C. until analysis. An amount of 50 μg of total of protein from each sample was used for determination of IgG levels using a mouse IgG ELISA kit (E-90G) from Immunology Consultants Laboratory, Inc. following the manufacturer's protocol.

Fluorometric immunocapture assay for MMP-9 activity: Enzymatic activity of MMP-9 in the ipsilateral cortex and striatum of WT and STEP KO mice subjected to 18 h reperfusion after MCAO was measured using a fluorescence resonance energy transfer (FRET) peptide immunoassay as described earlier (Hawkins et al., 2013). Briefly, high binding 96 well plates were first coated with protein A/G (200 μg/ml; ScienCell, Carlsbad, Calif.) in a carbonate/bicarbonate buffer (pH 9.4) for 2 h at room temperature. After washing the wells three times with TCNB buffer (200 μl each time, 50 mM Tris-HCl, 10 mM CaCl₂, 150 mM NaCl, 0.05% Brij L23) 0.5 μg of polyclonal rabbit anti-MMP-9 antibody (Cat. No. SC-6841-R, Santa Cruz Biotechnology) was added to each well and incubated for another 2 h. The wells were then washed again with TCNB buffer (three times) followed by addition of 50 μg of cortical and striatal lysates obtained from the ipsilateral hemisphere of WT and STEP KO mice and incubated overnight at 4° C. After washing with TCNB buffer 5-FAM/QXL™ 520 FRET peptide substrate (Substrate III Cat. No. AS-60570-01, Anaspec, Fremont, Calif.) was added to the wells. The plates were then incubated at 37° C. for 24 h, and then read at excitation/emission wavelengths of 485/528 nm in a Synergy HT multi-mode microplate fluorescence reader. Basal fluorescence was measured from wells where tissue lysates were not added and subtracted from the fluorescence values obtained from the sample treated wells to provide the final relative fluorescent unit (RFU) value for each sample.

Immunohistochemistry and quantification of Iba-1 fluorescence intensity: Six hours after MCAO/reperfusion mice were rapidly anesthetized and perfused intracardially with ice-cold 4% paraformaldehyde in 0.01M PBS. Brains were then removed and post-fixed in the same fixative solution for 4 h and cryoprotected in 15% and 30% sucrose in PBS and then frozen in Optical Cutting Temperature (OCT) compound. Brains were removed, cryoprotected and frozen for cryo-sectioning Immunohistochemistry with rabbit monoclonal anti-phospho-p38 (TPEYP) (Cat #4631), polyclonal anti-COX2 (1:100); monoclonal anti-Neun antibody from EMD Millipore (Cat #MAB-377); and polyclonal anti-Iba-1 antibody from Wako (Cat #019-19741) were performed on 16 μm sections. Briefly, sections were blocked with 10% normal goat serum, 3% BSA in PBS-T (PBS with 0.2% Triton X-100) for 1 h at room temperature and incubated overnight with the respective primary antibody. After extensive washing with PBS-T, sections were incubated with AlexaFluor-488 conjugated goat anti-rabbit antibody from Thermo Fisher (Cat #: A11008), AlexaFluor-488 conjugated goat anti-mouse antibody Thermo Fisher (Cat #: A11001) or Cy3 conjugated goat anti-rabbit antibody from Jackson Immuno-Research Laboratories (Cat #111-165-144) for 1 h at room temperature. Sections were washed three times in PBS-T and mounted using ProLong Gold Antifade mounting media. All sections were imaged using fluorescent microscopy (Olympus IX-71). To quantify microglial activation ImageJ (National Institutes of Health, Bethesda, Md., USA) was used to measure immunofluorescent staining intensity of Iba-1 in images captured from ischemic hemisphere of WT and STEP KO mice brain. Since Iba-1 is specifically expressed in microglia in the brain and is upregulated during the activation of this cells, increased intensity of Iba-1 immunofluorescence represents microgliosis (Ito et al., 2001; Patel et al., 2013; Yang et al., 2015).

Fluoro-Jade staining and infarct volume measurement: At the specified time points after MCAO (6, 12 and 24 h after reperfusion) or following sham surgery mice were anesthetized with sodium pentobarbital (150 mg/kg, i.p.) and perfused intracardially as described above and then frozen in OCT compound. Coronal forebrain sections were collected at 90 μm intervals. For Fluoro-Jade C staining sections were air-dried, dehydrated, incubated with 0.06% potassium permanganate (15 min), rinsed in water and stained with 0.001% Fluoro-Jade C (Histo-Chem) in 0.1% acetic acid for 30 min with gentle agitation on ice. Sections were rinsed in water, air-dried and mounted using Permount mounting media. Sections were then imaged using Olympus IX-71 fluorescent microscope as described earlier (Deb et al., 2013). In each slice, the total area in the contralateral side and the non-infarcted area in the lesioned side were measured by an investigator blinded to the experimental conditions using Image J software. The areas on each slide were summed over the numbers of section evaluated, and the respective volumes were calculated as follows: [(volume of contralateral side—non-infarcted volume of the lesioned side)/volume of contralateral side]×100% (Swanson et al., 1990).

Behavioral studies: Male mice treated with vehicle or STEP peptide mimetic (TAT-STEP-myc peptide) were subjected to neurological assessment and motor function tests (beam balance and rotarod) 24 h after MCAO (30 min) and reperfusion. Habituation to the testing environment and baseline training was performed one week before surgery. An observer blinded to the study groups and treatment conditions evaluated behavioral parameters after surgery. Severity of neurologic deficit (Longa et al., 1989) was assessed on a 5-point scale as follows: 0, no observable deficits; 1, failure to extend left forepaw; 2, circling to the left; 3, falling to left; 4, no spontaneous walking with a depressed level of consciousness; 5, death. For the rotarod test (Allan and Harris, 1989), mice were placed on an accelerating cylinder that rotated from 0 to 50 rpm over a period of 2 min. The time and speed at which the mice fell off the cylinder were measured automatically. For the beam balance test (Crabbe et al., 2003), mice were placed on a metal beam 1.3 cm diameter and 77 cm long, suspended 2 feet above the ground, and were required to traverse the beam. They were scored on a scale of 0-6 as follows: 0, cross the beam without any slips or hesitations; 1, cross the beam with 1 or 2 slips and/or hesitation; 2, cross the beam partially with multiple slips and falls; 3, balances with steady posture (>60 s) with multiple slips and 1 fall; 4, attempts to balance on the beam and falls off (>40 s) with multiple slips and falls but is walking on feet; 5, attempts to balance on the beam but falls off (>20 s), multiple falls and fails to walk on the feet; and 6, falls off: no attempt to balance or hang on the beam (>20 s).

Statistical analysis: Statistical differences between multiple groups were assessed using one-way ANOVA and Newman-Keuls post hoc analysis. For statistical differences between two groups, analysis was done using Student t-test. Data in the text and figures are expressed as mean±SEM. Mean differences between two groups were considered statistically significant when p<0.05.

### Results

Stimulation of Neurons with Glutamate in the Absence of STEP Leads to Sustained p38 MAPK Phosphorylation

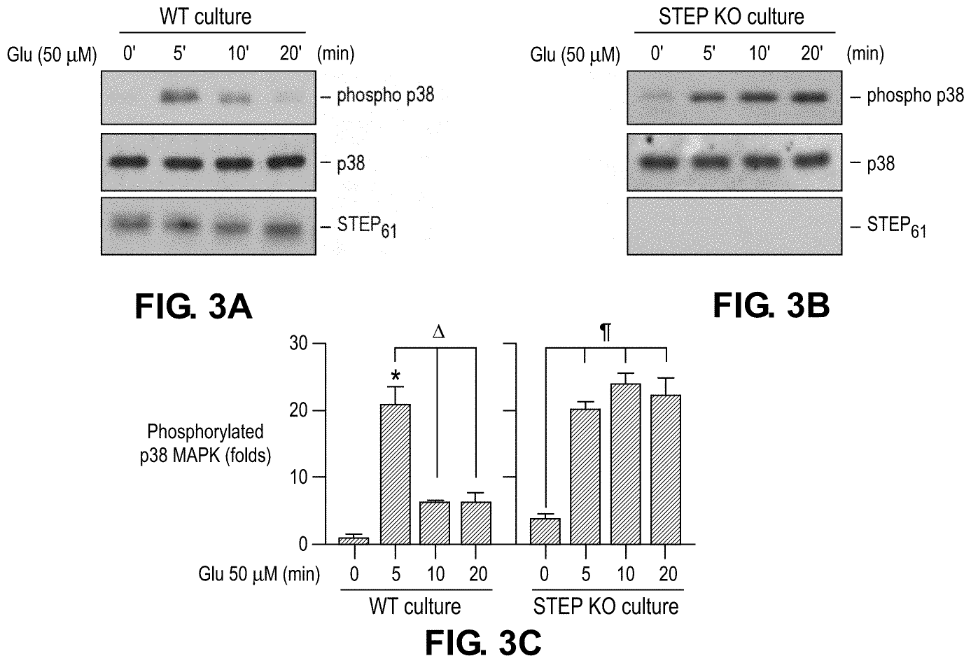

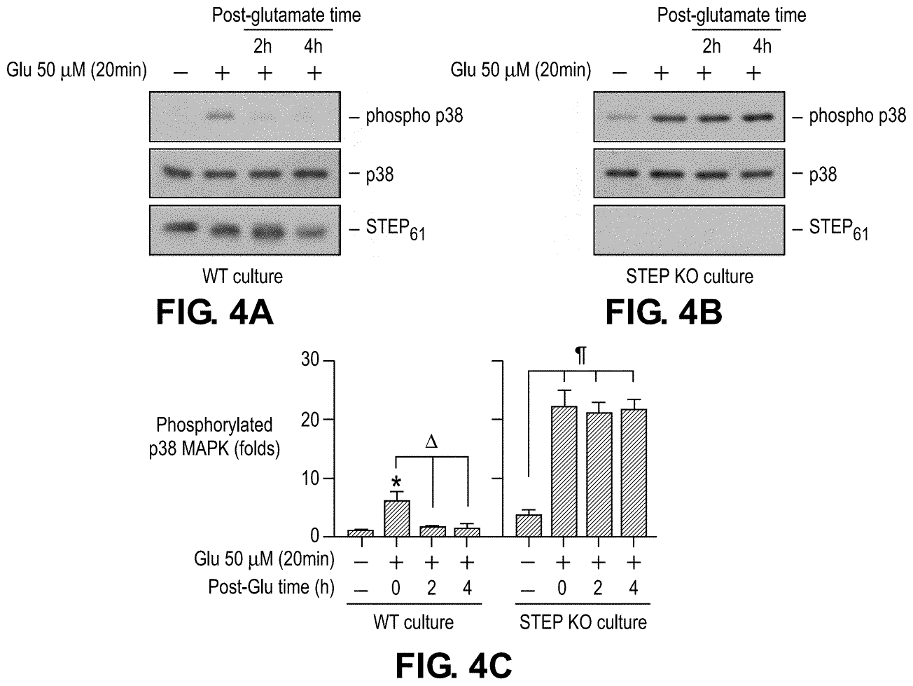

Our previous studies showed that p38 MAPK is a substrate of STEP (Poddar et al., 2010). FIGS. 3A-C show the results of corticostriatal neuronal cultures (12-14 days in vitro) from WT (FIG. 3A) and STEP KO (FIG. 3B) mice exposed to an excitotoxic insult to examine the possible consequences of deletion of endogenous STEP on p38 MAPK phosphorylation. For these experiments neuronal cultures were treated briefly with 50 μM glutamate (5, 10 or 20 min) without recovery to assess acute effects, or for 20 min followed by recovery (2 h and 4 h) to assess delayed effects of excitotoxicity. Cell lysate were processed for immunoblot analysis. Quantification of phosphorylated p38 MAPK in FIG. 3C is represented as mean±SEM. *, indicates significant difference from untreated control in WT cultures (p<0.05); ^{Δ}, indicates significant difference from 5 min glutamate treatment in WT cultures (p<0.05) and ^{¶}, indicates significant difference from untreated control in STEP KO cultures (p<0.05). FIGS. 4A-C show neuron cultures from WT (FIG. 4A) and STEP KO (FIG. 4B) mice treated with 50 μM glutamate (Glu) for 20 min and then maintained in its original medium for the specified times (post-glutamate time: 2 h and 4 h). Equal amount of protein from each sample was processed for immunoblot analysis using anti-phospho p38 MAPK (upper panel), -p38 MAPK (middle panel) or -STEP (lower panel) antibodies. Quantification of phosphorylated p38 MAPK (FIG. 4C) is represented as mean±SEM. *, indicates significant difference from untreated control in WT cultures (p<0.05); ^{Δ}, indicates significant difference from 20 min glutamate treatment in WT cultures (p<0.05) and ^{¶}, indicates significant difference from untreated control in STEP KO cultures (p<0.05).

As previously reported (Poddar et al., 2010), in neuronal lysates from WT mice a rapid increase in p38 MAPK phosphorylation was observed within 5 min of glutamate stimulation that decreased to near basal levels by 20 min of the insult (FIG. 3A). Analyzing the same samples with anti-STEP antibody showed STEP was phosphorylated in the untreated lysates (0 min), detected by an upward shift in the mobility of the STEP band. Glutamate exposure resulted in dephosphorylation and subsequent activation of STEP by 20 min, as evident from the downward shift in mobility of the STEP band (FIG. 3A, lower panel), which is consistent with earlier findings (Paul et al., 2003; Deb et al., 2013; Poddar et al., 2010). However, in neuronal lysates from STEP KO mice p38 MAPK phosphorylation remained sustained throughout the duration of the insult (FIG. 3B), and as expected no STEP protein was detectable (FIG. 3B lower panel). Additional studies showed that following recovery p38 MAPK phosphorylation in neuronal lysates from WT mice remained at basal levels (FIG. 4A, upper panel), although it remained elevated in the STEP KO mice neurons (FIG. 4B, upper panel). STEP also remained dephosphorylated in WT mice neurons during recovery (FIG. 4A, lower panel). These findings indicate that of loss of endogenous STEP could lead to prolonged activation of p38 MAPK signaling pathway in neurons following an excitotoxic insult.

Glutamate-Induced Increase in cPLA2 Activation, COX2 Expression and PGE₂ Level in STEP Deficient Neurons is Dependent on p38 MAPK Activation.

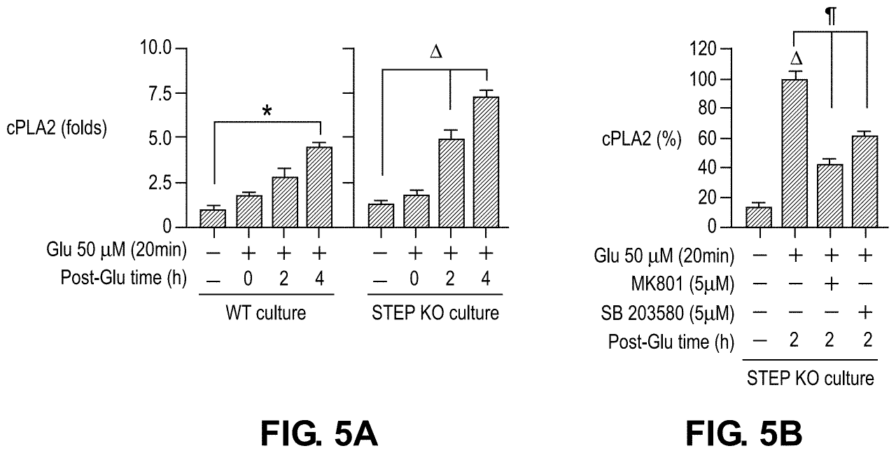

Earlier studies have indicated that excitotoxic stimulation of ionotropic glutamate receptor in neurons could activate cytosolic phospholipase-2 (cPLA2), which catalyzes membrane phospholipids to release arachidonic acid (AA), the initial substrate for PGE₂ biosynthesis (Minghetti 2004; Strauss and Marini 2006; Shen et al., 2007; Shelat et al., 2008). FIGS. 5A and B demonstrate the role of NMDA receptor and p38 MAPK in glutamate induced cPLA2 activation in neurons. In FIG. 5A, neuron cultures from WT or STEP KO mice were treated with 50 μM glutamate (Glu) for 20 min and then maintained in its original medium for the specified time (2 h and 4 h). In FIG. 5B, neuron cultures from STEP KO mice were treated with 50 μM glutamate (Glu) for 20 min in the presence or absence of MK801 or SB203580 and then maintained in the original medium in the presence of respective pharmacological inhibitors for the specified time period (2 h). Cell lysates with equal amount of protein from each sample was analyzed for cPLA2 activity using enzymatic assay. Values are expressed as mean±SEM. *, indicates significant difference from untreated control in WT cultures (p<0.05); ^{Δ}, indicates significant difference from untreated control in STEP KO cultures (p<0.05); ^{¶}, significant difference from 20 min glutamate treatment in STEP KO cultures (p<0.05). FIG. 5A shows a significant increase in cPLA2 activity in neuronal lysates from WT mice after 4 h of recovery following the insult. In neuronal lysates from STEP KO mice the basal cPLA2 activity remained unchanged when compared to WT controls. However, following glutamate treatment a significant increase in cPLA2 activity was observed within 2 h of recovery, which remained elevated over time. These results suggest that deletion of STEP gene accelerates glutamate-mediated cPLA2 activation in neurons. To determine the role of NMDARs and p38 MAPK in modulating cPLA2 activity in STEP KO mice cultures, neurons were treated with glutamate (50 μM, 20 min) in the presence of NMDAR inhibitor MK801 (5 μM) or p38 MAPK inhibitor SB 203580 (5 μM). FIG. 5B shows that application of either MK801 or SB 203580 during the insult significantly reduced cPLA2 activity observed 2 h after the insult.

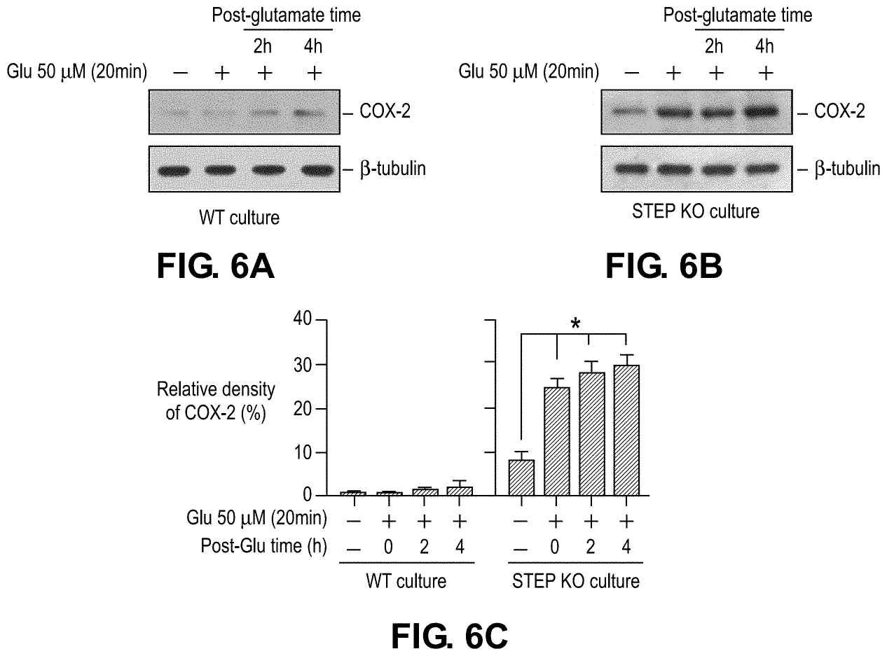

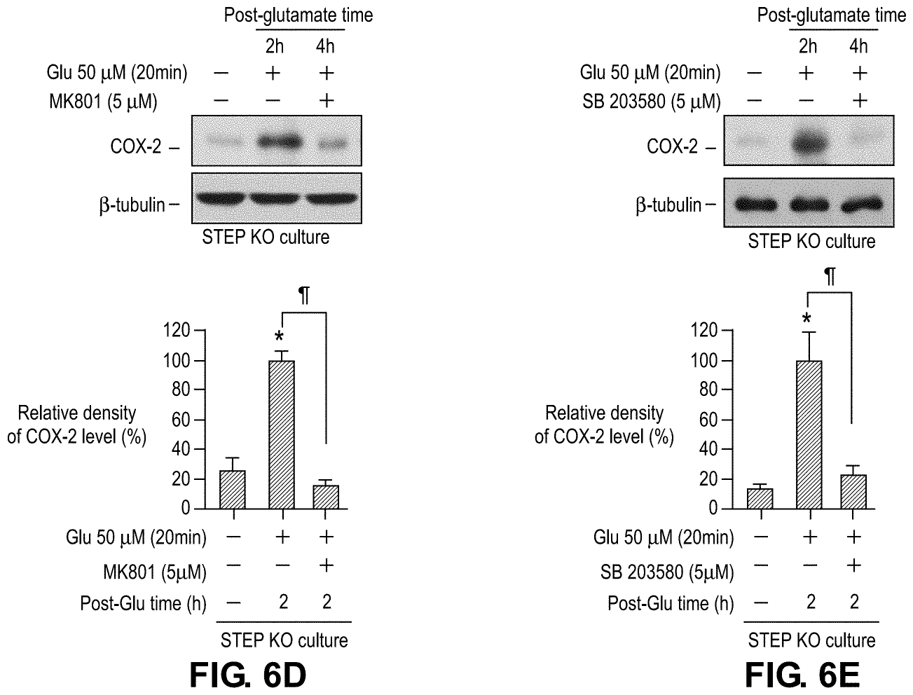

In a parallel series of experiments, we further evaluated the expression of COX2, which is involved in the conversion of AA to prostanoids. FIGS. 6A-E demonstrate the role of NMDA receptor and p38 MAPK in glutamate induced increase in COX2 protein level in neurons. Neuron cultures from WT (FIG. 6A) and STEP KO (FIG. 6B) mice were treated with 50 μM glutamate (Glu) for 20 min and then maintained in its original medium for the specified time (post-glutamate time: 2 h and 4 h). Neuron cultures were treated with 50 μM glutamate (Glu) for 20 min in the absences or presence of MK801 (FIG. 6D) or SB203580 (FIG. 6E) and then maintained in the original medium in the presence of the respective pharmacological inhibitors for the specified time period (post-glutamate time: 2 h). Cell lysates with equal amount of protein from each sample was analyzed by immunoblotting with anti-COX2 (upper panel) or anti-β-tubulin (lower panel) antibodies. Quantification of COX2 is represented as mean±SEM. *, indicates significant difference from untreated control in STEP KO cultures (p<0.05); ^{¶}, indicates significant difference from glutamate treated STEP KO cultures (p<0.05). FIG. 6A (upper panel) shows that the COX2 level in neurons from WT mice remained unchanged during the insult or during recovery when compared to untreated control. However, in the neurons from the STEP KO mice COX2 level increased significantly within 20 min of the insult and remained elevated during the recovery phase (FIG. 6B, upper panel). Pharmacological studies further showed that inhibition of NMDAR with MK801 or p38 MAPK activation with SB 203580 attenuated glutamate-induced increase in COX2 level in the STEP KO mice neurons (FIG. 6C, D).

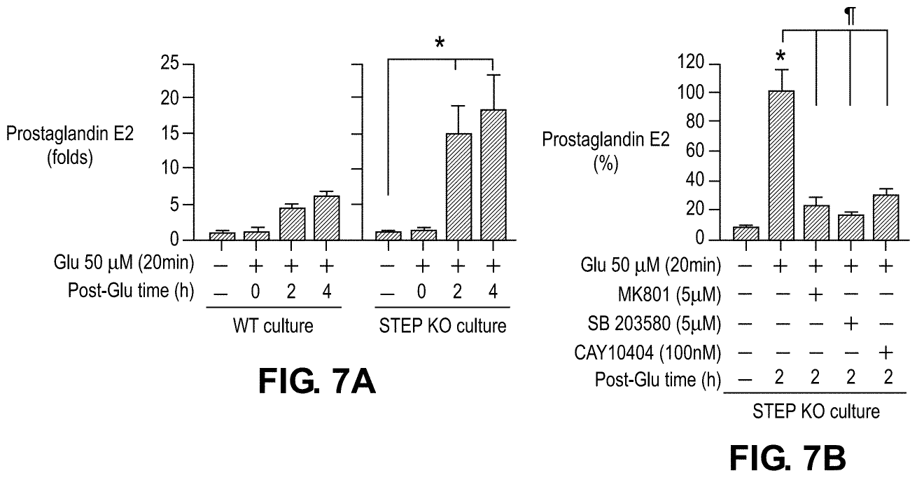

To evaluate the effect of STEP gene deletion on glutamate-mediated PGE₂ release in subsequent studies neuron cultures from WT or STEP KO mice were treated with 50 μM glutamate (Glu) for 20 min and then maintained in its original medium for the specified times (post-glutamate time: 2 h and 4 h). (FIG. 7A) In FIG. 7B, neuron cultures from STEP KO mice were treated with 50 μM glutamate (Glu) in the absence or presence of MK801, SB203580 or CAY10404 and then maintained in the original medium in the presence of the respective pharmacological inhibitors for the specified time period (2 h). Equal amount of culture medium from each sample was analyzed for PGE₂ levels using ELISA. Values are expressed as mean±SEM. *, indicates significant difference from untreated control in STEP KO cultures (p<0.05); ^{¶}, indicates significant difference from glutamate treated STEP KO cultures (p<0.05). The results showed that glutamate treatment had no significant effect on PGE₂ release from WT mice neurons. However, PGE₂ release increased significantly from STEP KO mice neurons within 2 h of the recovery phase, which remained elevated thereafter (FIG. 7A). Additional studies investigated the effect of NMDAR inhibition, p38 MAPK inhibition as well as COX2 inhibition (CAY10404, 100 nM) in glutamate-mediated PGE₂ release from STEP KO mice neurons. FIG. 7B shows that glutamate-induced PGE₂ release from STEP KO mice neurons is significantly reduced following inhibition of NMDAR, p38 MAPK and COX2. These findings strongly suggest the involvement of p38 MAPK-COX2 signaling pathway in glutamate NMDAR induced neuronal PGE₂ release in the absence of STEP.

Glutamate-Induced Increase in COX-2 Expression and PGE₂ Release in the Absence of STEP Involves p38 MAPK Mediated cPLA2 and NFκB Activation.

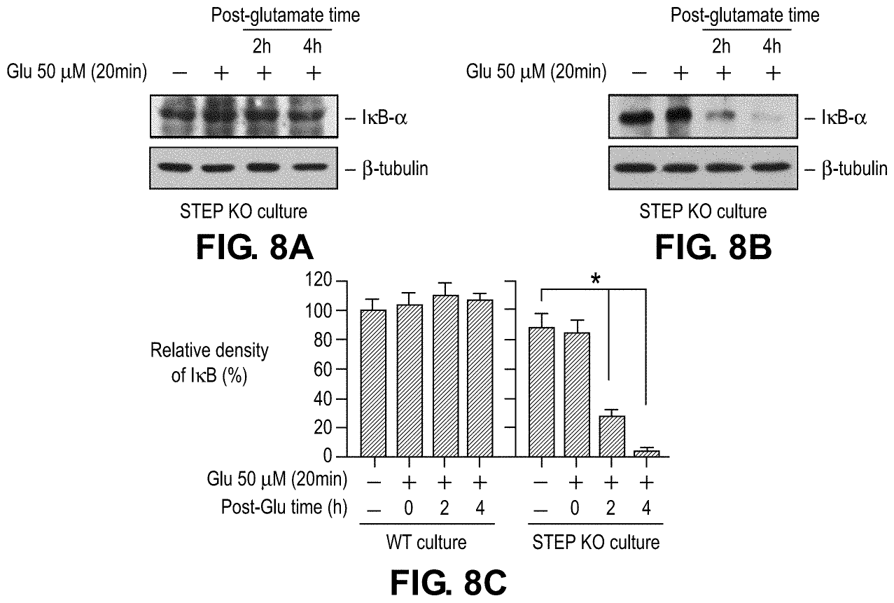

Earlier studies have shown that the nuclear factor kappa B (NF-κB) is a key regulator of COX2 expression in different cell types (Yamamoto et al., 1995; Kaltschmidt et al., 2002; Guo et al., 2013; Ackerman et al., 2008; Shi et al., 2015), and the ionotropic glutamate receptors is one of the activators of NF-κB in neurons (Kaltschmidt et al., 2005). To clarify the role of NF-κB in glutamate-NMDAR induced activation of the COX-2-PGE₂ signaling pathway in STEP KO mice neurons, we next evaluated the effect of glutamate on IκB degradation, a seminal step in NF-κB activation (Karin and Ben-Neriah 2000; Rajagopal et al., 2019). Neuron cultures from WT (FIG. 8A) and STEP KO (FIG. 8B) mice were treated with 50 μM glutamate (Glu) for 20 min and then maintained in its original medium for the specified time (post-glutamate time: 2 h and 4 h). Cell lysates with equal amount of protein from each sample was analyzed by immunoblotting with anti-IκB-α (upper panel) and anti-β-tubulin (lower panel) antibodies. In FIG. 8C, Quantification of IκB-α level is represented as mean±SEM. *, indicates significant difference from untreated control in STEP KO culture (p<0.05) Immunoblot analysis of neuronal lysates showed that brief exposure to glutamate had no effect on IκB level as compared to untreated control, in the presence or absence of STEP. However, in neuronal lysates from STEP KO mice a significant decrease in IκB level was observed at 2 h and 4 h following the 20 min of insult, indicating IκB degradation and NF-κB activation.

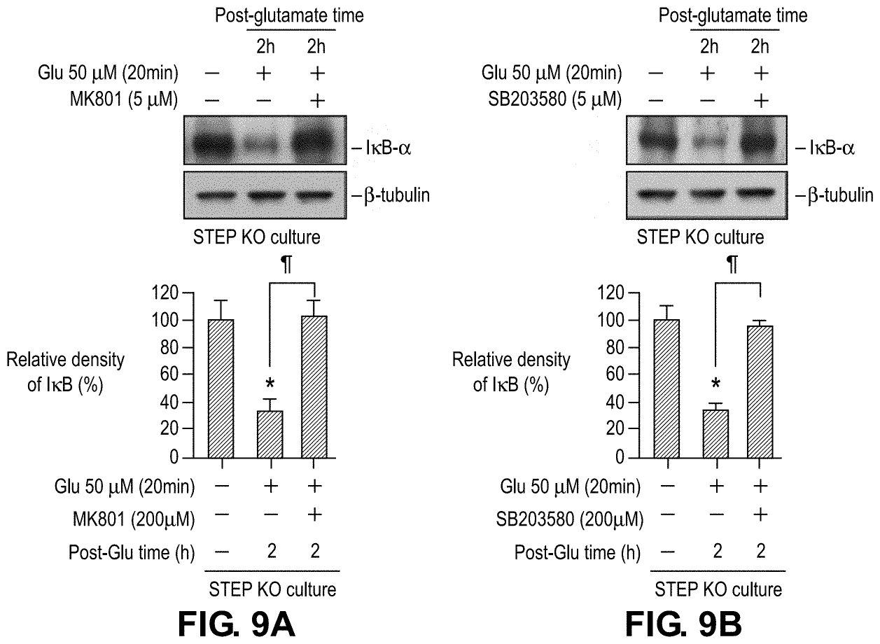

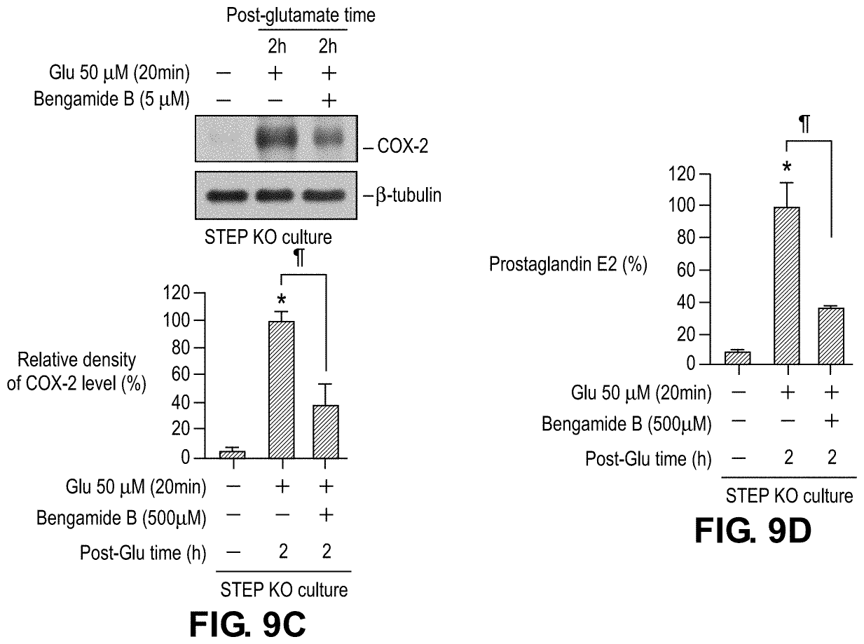

FIGS. 9A-9D show the role of NFκB activation in glutamate induced COX2 expression and PGE₂ release. Neuron cultures from STEP KO mice were treated with 50 μM glutamate (Glu) in the absence or presence of (FIG. 9A) MK801, (B) SB203580 or (FIG. 9C, D) Bengamide B and then maintained in the original medium in the presence of the respective pharmacological inhibitors for the specified time period (2 h). (FIG. 9A-C) Cell lysates with equal amount of protein from each sample was analyzed by immunoblotting with (FIG. 9A, B) anti-IκB-α (upper panel) and anti-β-tubulin (lower panel) antibodies, or (C) anti-COX-2 (upper panel) and anti-β-tubulin (lower panel) antibodies. Quantification of IκB-α and COX-2 are represented as mean±SEM. *, indicates significant difference from untreated control in STEP KO culture (p<0.05); ^{¶}, indicates significant difference from glutamate treated STEP KO culture. (FIG. 9D) Equal amount of culture medium from each sample was analyzed for PGE₂ levels using ELISA. Values are expressed as mean±SEM. *, indicates significant difference from untreated control in STEP KO culture (p<0.05); ^{¶}, indicates significant difference from glutamate treated STEP KO culture (p<0.05).

As shown in FIGS. 9A-D, exposure to glutamate in the presence of NMDAR inhibitor MK801 or p38 MAPK inhibitor effectively blocked glutamate-induced IκB degradation (FIG. 9A, B). In additional studies (FIG. 9C) neurons were treated with glutamate in the presence of Bengamide B (500 nM), a potent inhibitor of NF-κB activation (Rajagopal et al., 2019). Immunoblot analysis showed that co-incubation with Bengamide B attenuated glutamate induced increase in COX2 protein level. Evaluation of PGE₂ release in the culture medium obtained from the same experiments showed significant reduction in PGE₂ release following exposure to glutamate (FIG. 9D).

Restoration of STEP Signaling with a STEP-Peptide Mimetic Attenuates Glutamate-Induced Attenuates p38 MAPK Activation, COX2 Expression and PGE₂ Release.

To directly test the hypothesis that upregulation p38 MAPK-COX-2 signaling pathway in the absence of endogenous STEP leads to increased PGE₂ release following exposure to glutamate, we generated a cell-permeable a TAT-STEP-myc peptide (FIG. 10) that constitutively binds to and inhibits p38 MAPK activation in neurons (Poddar et al., 2010).

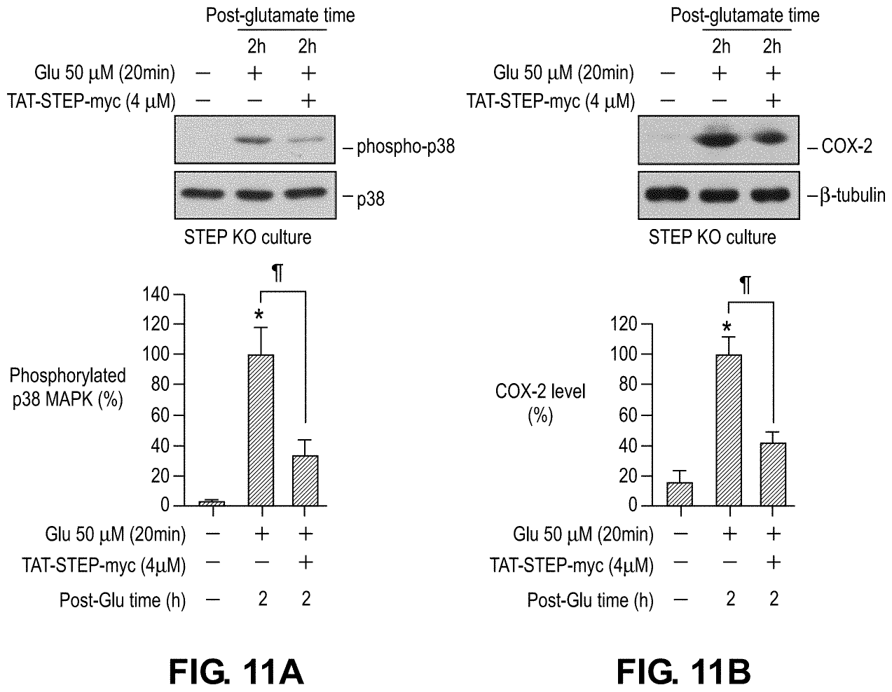

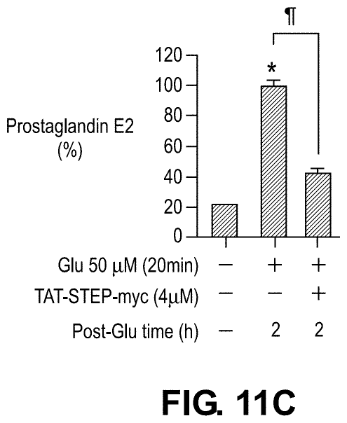

FIGS. 11A-C show the effect of the STEP peptide mimetic (TAT-STEP-myc) shown in FIG. 10 on glutamate-induced increase in p38 MAPK phosphorylation (FIG. 11A), COX2 expression (FIG. 11B) and PGE₂ release (FIG. 11C) from STEP KO mice neurons. Neuron cultures from STEP KO mice were treated with 50 μM glutamate (Glu) in the absence or presence of TAT-STEP-myc peptide and then maintained in the original medium in the presence of the peptide for the specified time period (2 h). Cell lysates with equal amount of protein from each sample was analyzed by immunoblotting using anti-phospho p38 MAPK (upper panel) or -p38 MAPK (lower panel) antibodies (FIG. 11A), or anti COX2 (upper panel) and anti-β-tubulin (lower panel) antibodies (FIG. 11B). Quantification of phosphorylated p38 MAPK and COX2 levels are represented as mean±SEM. In FIG. 11C, an equal amount of culture medium from each sample was analyzed for PGE₂ levels using ELISA. Values are expressed as mean±SEM. *, indicates significant difference from untreated control in STEP KO culture (p<0.05); ^{¶}, indicates significant difference from glutamate treated STEP KO culture (p<0.05).

Immunoblot analysis showed that peptide application blocked the phosphorylation of p38 MAPK assessed 2 h after a 20 min glutamate exposure (FIG. 11A). COX2 expression was also significantly reduced following peptide treatment (FIG. 11B). Evaluation of PGE₂ release in the medium 4 h after stimulation showed significant reduction in PGE₂ release (FIG. 11C). These findings highlight the role of STEP in regulating a proinflammatory response in neurons following an excitotoxic insult.

Increase in Neuronal p38 MAPK Phosphorylation, COX-2 Expression and PGE₂ Release in STEP Deficient Mice after Ischemic Insult.

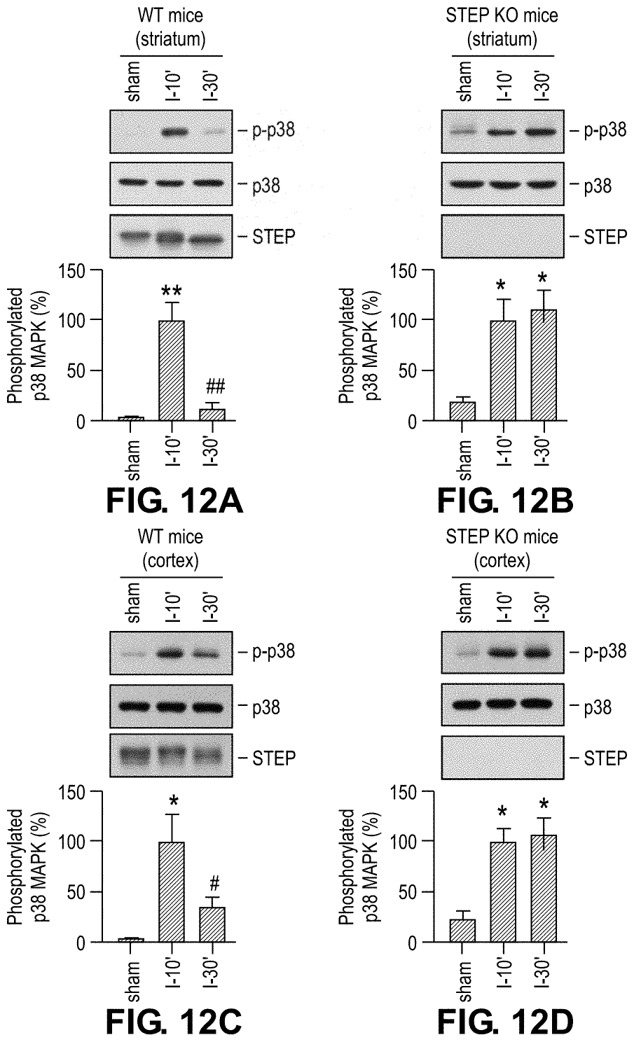

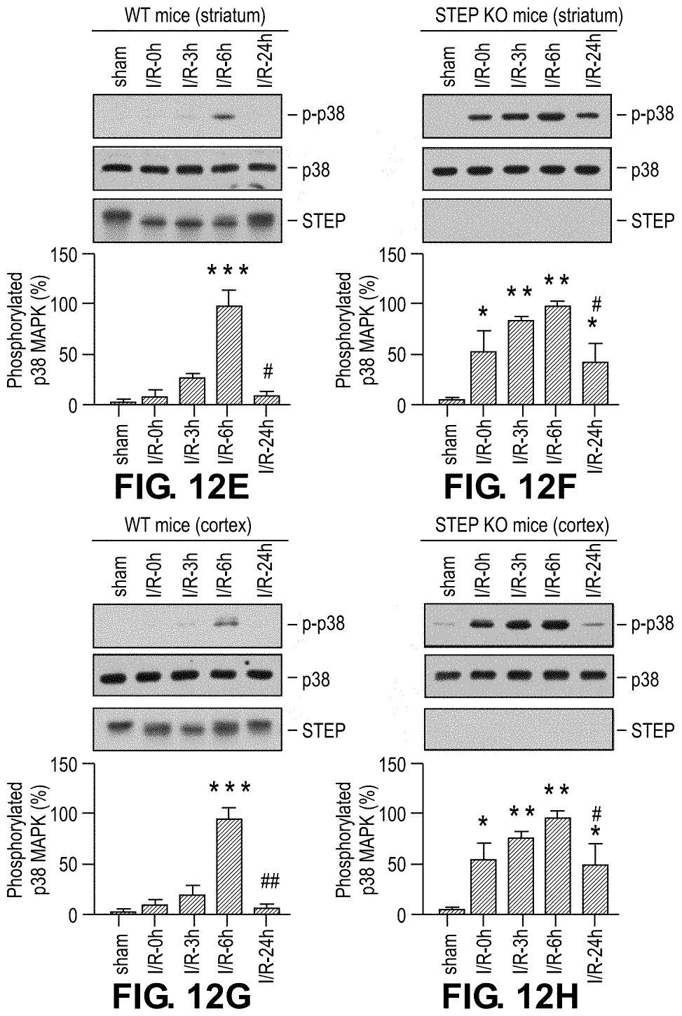

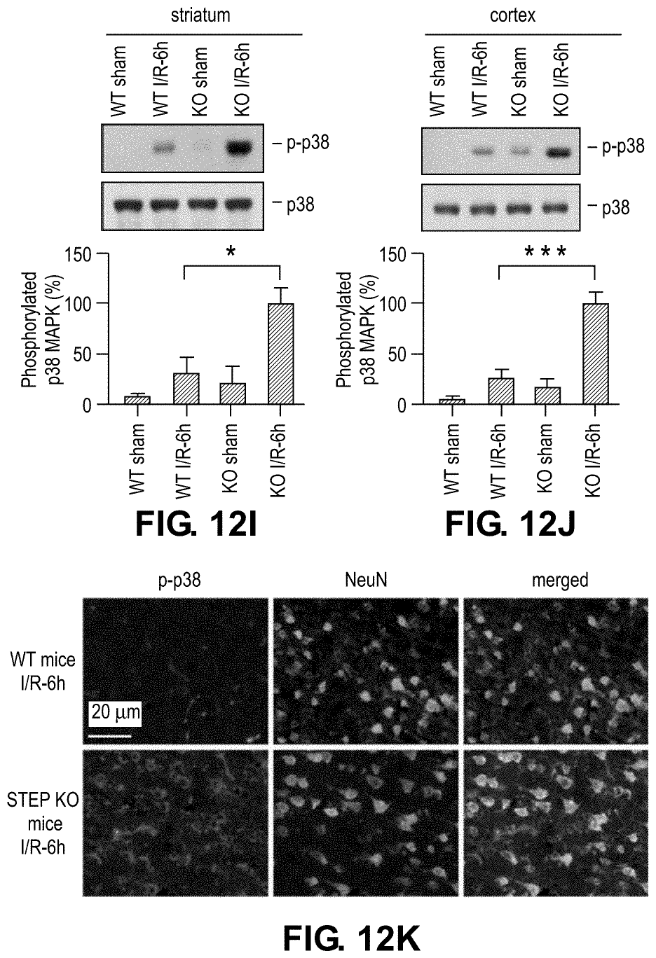

Among the known kinases that are regulated by STEP, p38 MAPK is active in ischemic condition (Barone et al., 2001a; Barone et al., 2001b; Nozaki et al., 2001). To test whether a transient focal ischemia in the absence of STEP has any effect on p38 MAPK phosphorylation, WT and STEP KO mice were subjected to MCAO for varying time periods (10 or 30 min). FIGS. 12A-K demonstrate a sustained increase in p38 MAPK phosphorylation in STEP KO mice during ischemia and reperfusion. WT (FIGS. 12A and 12C) and STEP KO (FIGS. 12B and 12D) mice were subjected to sham surgery or MCAO (FIG. 12I) for the specified time periods (10 and 30 min). WT (FIGS. 12E and 12G) and STEP KO (FIGS. 12F and 12H) mice were subjected to sham surgery or MCAO (FIG. 12I) for 30 min followed by reperfusion (R) for the specified time periods (0, 3, 6 and 24 h). Tissue extracts with equal amount of protein from the ipsilateral (FIGS. 12A, B, E, F) striatum and (FIGS. 12 C, D, G, H) cortex was analyzed with anti-phospho-p38 MAPK (upper panels). Equal protein loading in each sample was confirmed by re-probing the blots with anti-p38 MAPK antibody (middle panels). STEP was analyzed by immunoblotting with anti-STEP (lower panels) antibody. The increase in p38 MAPK phosphorylation in the (FIG. 12I) striatum and cortex (FIG. 12J) of WT and STEP KO mice at 6 h post-MCAO reperfusion time point was compared by immunoblot analysis using anti-phospho-p38 MAPK antibody (upper panels) and anti-p38 MAPK antibody (lower panels). FIG. 12A-D Bar diagrams represent mean±SEM (n=4-5 mice/group). *p<0.05 and **p<0.001 from sham; ^{#}p<0.05 and ^{##}p<0.001 from MCAO 10 min (I-10′). FIG. 12E-J Bar diagrams represent mean±SEM (n=3-4 mice/group. FIG. 12E-H *p<0.01, **p<0.001 and ***p<0.0001 from sham; ^{#}p<0.01 and ^{##}p<0.0001 from 6 h post-ischemic reperfusion (I/R-6 h). FIG. 12I, J *p<0.01 and ***p<0.0001 from WT I/R-6 h. (K) WT and STEP KO mice were subjected to 30 min MCAO followed by reperfusion for 6 h and then processed for immunohistochemistry with anti-phospho-p38-MAPK and NeuN antibodies.

Immunoblot analysis shows a rapid increase in p38 MAPK phosphorylation in both striatum and cortex of WT (FIG. 12A, C) and STEP KO mice (FIG. 12B, D) within 10 min of the insult. However, in the WT mice p38 MAPK phosphorylation decreases to near basal level by 30 min in both striatum and cortex, while it remains sustained in STEP KO mice. In WT mice, ischemia also leads to de-phosphorylation and subsequent activation of STEP by 30 min as evident from the downward shift in the mobility of the STEP band (FIG. 12A, C), which is consistent with our earlier observation in both cell culture model of excitotoxicity and in animal models of ischemia (Deb et al., 2013; Poddar et al., 2010). As expected, the STEP protein band is not detectable in the tissue lysates from STEP KO mice (FIG. 12B, D). Subsequent studies evaluated the effect of reperfusion after 30 min MCAO, on p38 MAPK phosphorylation in both WT and STEP KO mice. FIGS. 12E and 12G show that in the WT mice, p38 MAPK phosphorylation remains at basal levels both at the onset of reperfusion (I/R-0 h) as well as 3 h after the onset of reperfusion (I/R-3 h), and increases only transiently at 6 h after reperfusion (I/R-6 h). This secondary activation of p38 MAPK in WT mice is associated with inactivation of STEP through phosphorylation, as evident from the upward shift in the mobility of the STEP band. In contrast, p38 MAPK phosphorylation remains continuously elevated in STEP KO mice for at least 6 h after the onset of reperfusion (FIGS. 12F, H). A comparative analysis of the magnitude of p38 MAPK phosphorylation at 6 h post-ischemic reperfusion shows a significantly large increase in p38 MAPK phosphorylation in both striatum (—˜3-fold) and cortex (˜4-fold) of STEP KO mice, when compared to WT littermates (FIGS. 12I, J). Immunohistochemical staining of coronal brain sections with anti-phospho-p38 MAPK and NeuN antibodies further show that the increase in p38 MAPK phosphorylation in STEP KO mice is localized primarily in neurons (FIG. 12K).

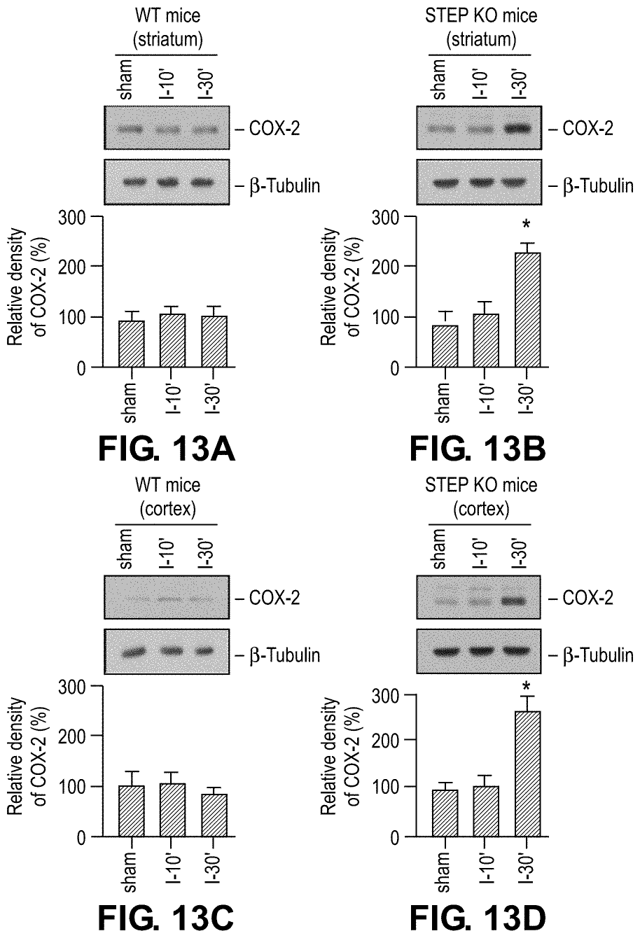

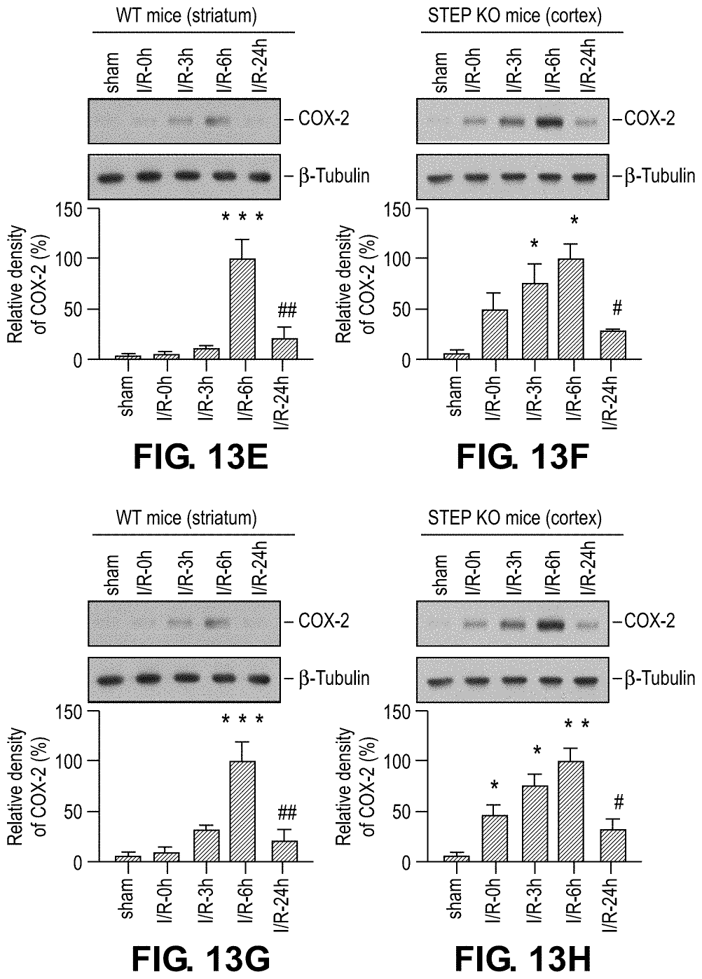

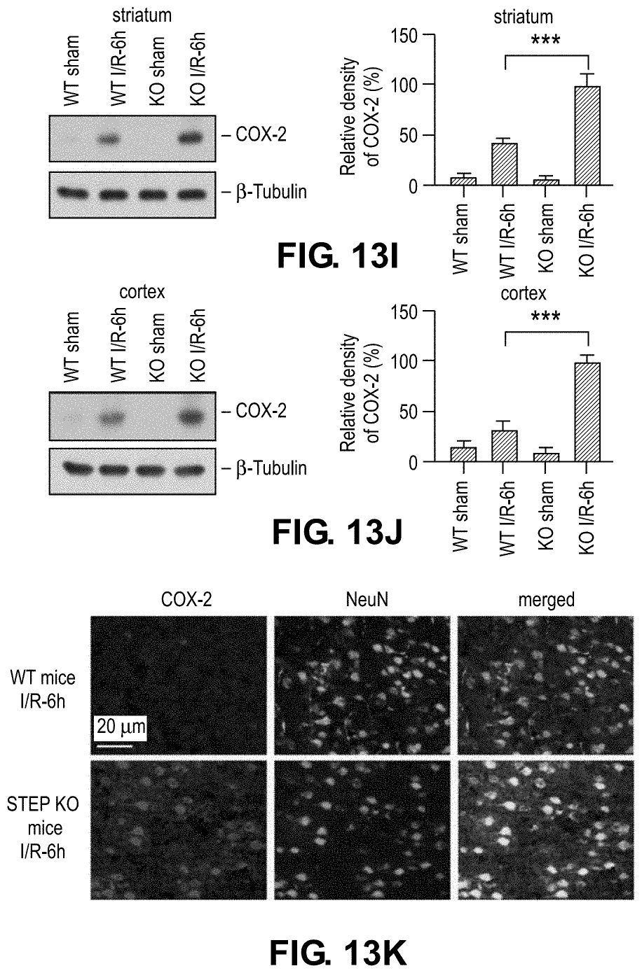

Emerging evidence indicate that depending on the cell type and stimuli, p38 MAPK can enhance transcription and/or stability of COX-2, an enzyme that catalyzes the conversion of arachidonic acid into inflammatory prostaglandins in the post-ischemic brain (Miettinen et al., 1997; Nito et al., 2008; Nogawa et al., 1997; Sasaki et al., 2004; Smith et al., 2000). Therefore, we next investigated the expression of COX2 in both WT and STEP KO mice, during the ischemic insult and reperfusion. The results are shown in FIGS. 13A-13M. WT (FIGS. 13A, C) and STEP KO (FIGS. 13B, D) mice were subjected to sham surgery or MCAO (FIG. 13I) for the specified time periods (10 and 30 min). (FIGS. 13E, G) WT and (FIGS. 13F, H) STEP KO mice were subjected to sham surgery or MCAO (FIG. 13I) for 30 min followed by reperfusion (R) for the specified time periods (0, 3, 6 and 24 h). COX2 protein levels in tissue lysates from the ipsilateral (FIGS. 13A, B, E, F) striatum and (FIGS. 13C, D, G, H) cortex was analyzed with anti-COX2 antibody (upper panels). Equal protein loading in each sample was confirmed by re-probing the blots with anti-β-tubulin antibody (lower panels). (FIGS. 13I, J) The increase in COX2 level in the striatum and cortex of WT and STEP KO mice at 6 h post-MCAO reperfusion time point was compared by immunoblot analysis, using anti-COX2 antibody (upper panels) and anti-β-tubulin antibody (lower panels). In FIGS. 13A-D, bar diagrams represent mean±SEM (n=4 mice/group). *p<0.01 from sham. In FIGS. 13 E-J, bar diagrams represent mean±SEM (n=3-5 mice/group). (E-H) *p<0.05, **p<0.001 and ***p<0.0001 from sham; ^{#}p<0.05 and ^{##}p<0.001 from 6 h post-ischemic reperfusion (I/R-6 h). (I, J) ***p<0.0001 from WT I/R-6 h. In FIG. 13K, WT and STEP KO mice were subjected to 30 min MCAO followed by reperfusion for 6 h and then processed for immunohistochemistry with anti-COX2 and NeuN antibodies. WT (FIG. 13L) and STEP KO (FIG. 13M) mice were subjected to sham surgery or 30 min MCAO followed by reperfusion for 6 h. PGE₂ level was measured by enzyme immunoassay in the supernatants obtained from ipsilateral striatum and cortex of both WT and STEP KO mice. Data are expressed as nanograms (ng) of PGE₂ level per milligram (mg) of cellular protein. Bar diagram represent mean±SEM (n=3-4 mice/group) and ***p<0.0001 between WT I/R 6 h and KO I/R 6 h.

During the ischemic insult in the WT mice, COX2 expression remain unchanged as compared to the sham-operated control mice (FIG. 13A, C), while in the STEP KO mice, COX2 expression increases significantly within 30 min of the insult in the ipsilateral striatum and the cortex (FIGS. 13B, D). During reperfusion, a transient increase in COX2 expression is observed 6 h after post-ischemic reperfusion in the WT mice (FIGS. 13E, G). In contrast, COX2 levels in the STEP KO mice increases progressively from the onset of reperfusion through 6 h of post-ischemic reperfusion in both the ipsilateral striatum and cortex (FIGS. 13F, H). Comparison of COX2 protein levels between WT and STEP KO mice at 6 h of post-ischemic reperfusion shows significant increase in COX2 level in both the striatum (˜2-fold) and cortex (˜3-fold) of STEP KO mice (FIGS. 13I, J) Immunohistochemical staining further show that the up regulation of COX2 protein levels in STEP KO mice at 6 h of post-ischemic reperfusion is specifically in neurons (FIG. 13K). PGE₂ immunoassay show that the elevated COX2 protein level in the STEP KO mice is associated with significantly higher levels of the inflammatory PGE₂ in both the ipsilateral striatum and cortex of STEP KO mice brain when compared with WT littermates (FIGS. 13L, M). The elevated COX2 expression in neurons and the enhanced PGE₂ production strongly suggests the involvement of neuronal PGE₂ release in increasing post-ischemic inflammatory response in the absence of endogenous STEP.

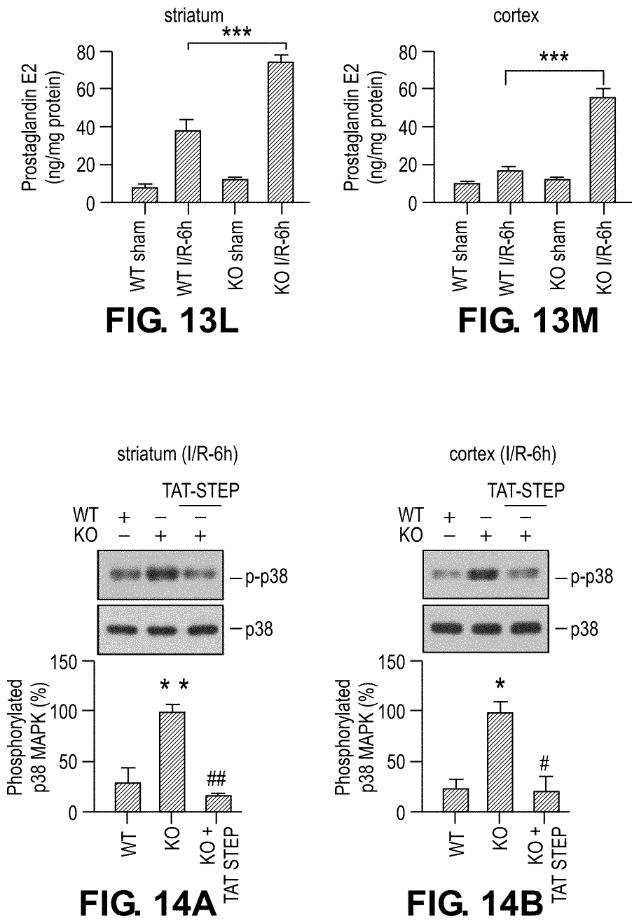

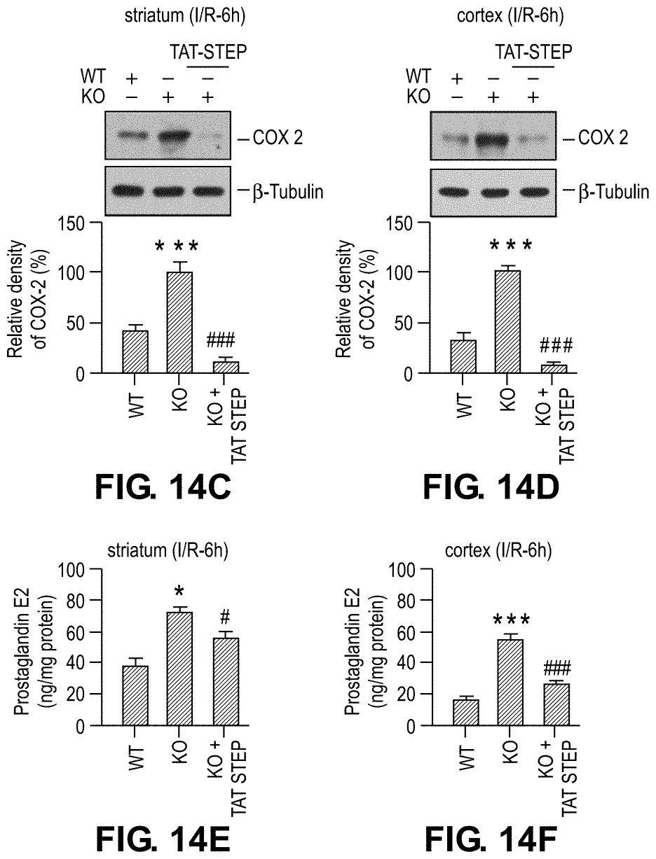

FIGS. 14A-F demonstrate that the STEP peptide mimetic attenuates post-ischemic increase in p38 MAPK phosphorylation, COX2 expression and PGE₂ release in STEP KO mice. WT and STEP KO mice were subjected to MCAO (30 min) and reperfusion (6 h). The STEP peptide mimetic (TAT-STEP-Myc) (FIG. 10) was administered in a subset of STEP KO mice at the onset of reperfusion (KO+TAT-STEP). p38 MAPK phosphorylation (FIGS. 14A, B) and COX2 protein level (FIGS. 14C, D) were evaluated by immunoblot analysis of tissue extracts from ipsilateral striatum (FIGS. 14A, C) and cortex (FIGS. 14B, D) obtained from WT and STEP KO mice. Blots were re-probed with anti-p38 MAPK (FIGS. 14A, B)) or anti tubulin antibody (FIGS. 14C, D) (lower panels). The corresponding bar diagrams represents mean±SEM (n=4-6 mice/group). In FIGS. 14E, F, PGE₂ level was measured by enzyme immunoassay in the supernatants obtained from ipsilateral striatum and cortex (expressed as ng/mg protein). In FIGS. 14A-F, bar diagrams represent mean±SEM (n=4-6 mice/group). *p<0.05, **p<0.001 and ***p<0.0001 from WT; ^{#}p<0.05, ^{##}p<0.001 and ^{###}p<0.001 from KO.

To determine whether loss of STEP is a key factor in the up regulation of neuronal p38 MAPK-COX2-PGE₂ signaling pathway under ischemic condition, STEP KO mice were subjected to MCAO (30 min) followed by intravenous administration of a single dose of the STEP-peptide mimetic, TAT-STEP-Myc (3 nmol/g, FIG. 10) at the onset of reperfusion. FIGS. 14A-F show that restoration of STEP signaling significantly reduced p38 MAPK phosphorylation in both the striatum and cortex of STEP KO mice when treated with the peptide and observed 6 h after reperfusion (FIGS. 14A, B). Evaluation of COX2 protein level (FIGS. 14C, D) and PGE₂ level (FIGS. 14E, F) at 6 h after reperfusion also show significant reduction in the peptide treated mice.

Increased Microglial Activation in STEP Deficient Mice after Ischemic Insult.

Excessive or persistent release of PGE₂ in the brain has been associated with the activation of microglia that are known to participate in the progression of ischemic pathology (del Zoppo et al., 2007; Kreutzberg, 1996; Mabuchi et al., 2000; Wang et al., 2007). Activated microglia are defined partly by their change in morphology from a ramified state characterized by a small body and multiple thin processes to a more amoeboid shape with highly branched short processes and increased immunoreactivity for Iba-1 (Ito et al., 2001; Zhang et al., 1997).

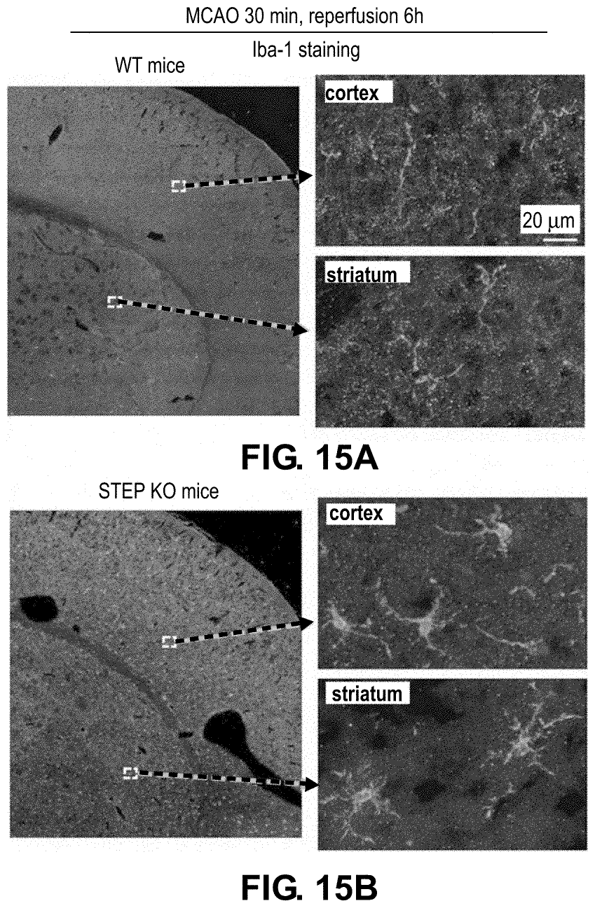

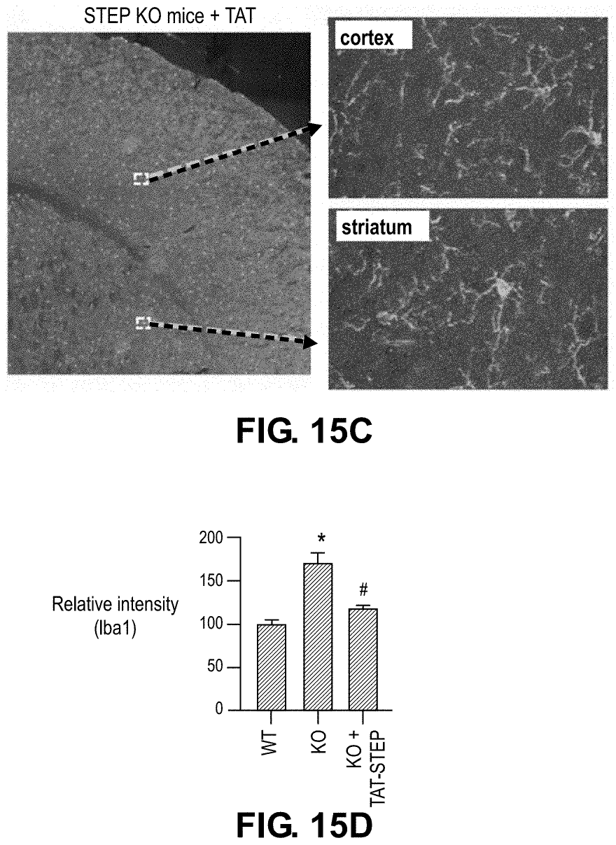

To investigate whether microglia in STEP KO mice are affected by transient MCAO (30 min) and reperfusion, the morphology of cells immune-reactive for Iba-1 were evaluated in both WT and STEP KO mice brain sections 12 h after reperfusion. FIGS. 15A-D demonstrate that the STEP peptide mimetic attenuates microglial activation in the post-ischemic brain of STEP KO mice. WT and STEP KO mice were subjected to MCAO (30 min) and reperfusion (6 h). In a subset of STEP KO mice, the STEP peptide mimetic (TAT-STEP-Myc) (FIG. 10) was administered at the onset of reperfusion (KO+TAT-STEP) Immunohistochemical staining with anti-Iba-1 antibody show coronal brain sections through cortex and striatum of the ipsilateral hemisphere (FIGS. 15A-C, left panels). The right panels present higher magnification of the images shown in squares in the left panels. The images in the right panels show changes in microglial morphology (ramified, inactive form or amoeboid, active form), in both the striatum and cortex. FIG. 15D shows quantification of immunofluorescent staining intensity of Iba-1 (relative intensity) in the striatum and cortex of the ischemic hemisphere. Values represent mean±SEM (n=3 mice/group). *p<0.001 from WT and ^{#}p<0.001 from KO.

Representative microglial phenotype presented in FIGS. 15A-D indicate that change in microglial morphology is not so apparent in either the striatum or cortex of WT mice (FIG. 15A), whereas in the STEP KO mice distinct changes in microglial morphology to the amoeboid form is observed in both the ipsilateral striatum and cortex (FIG. 15B). Quantification of immunofluorescent staining intensity further show a significant increase in immunoreactivity for Iba-1 in the brain of STEP KO mice, when compared with WT littermates (FIG. 15D). Additional studies indicate that administration of the STEP peptide mimetic at the onset of reperfusion blocks change in microglial morphology to the amoeboid state (FIG. 15C) and significantly reduces Iba-1 immunoreactivity (FIG. 15D).

Increased MMP-9 Activity and BBB Permeability in STEP Deficient Mice after Ischemic Insult

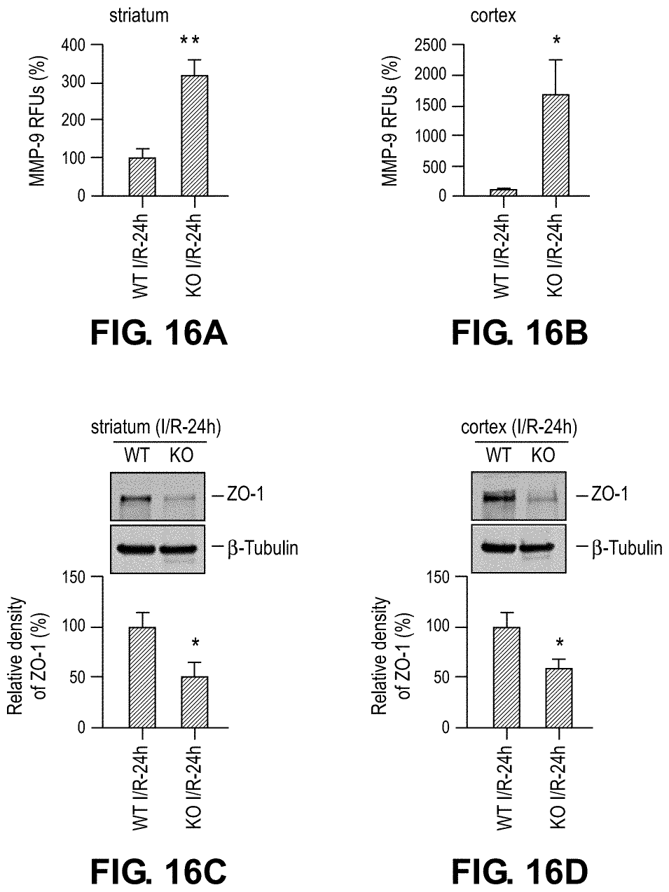

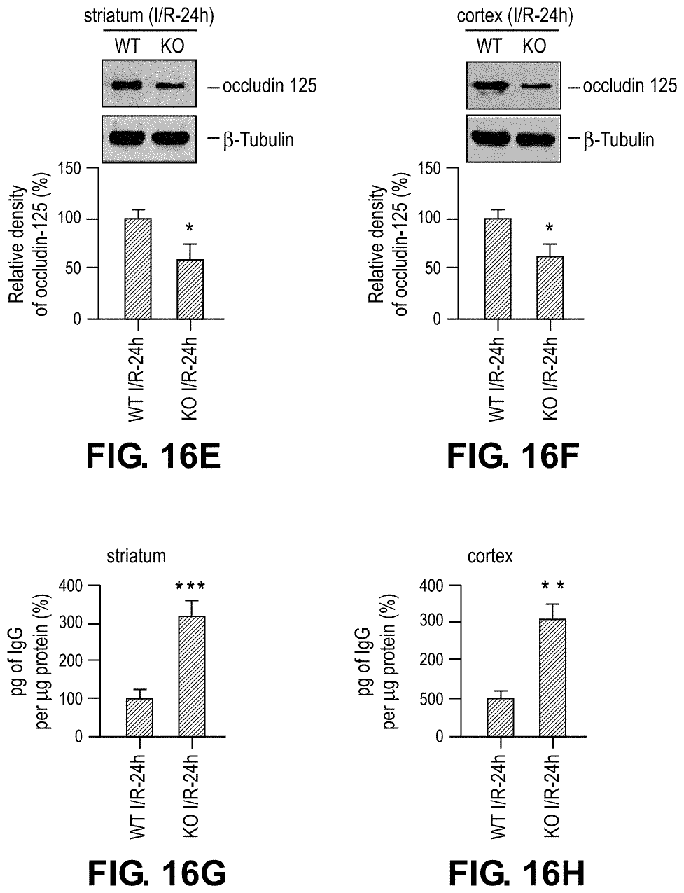

Given that the level and activity of the proteolytic enzyme MMP-9 and subsequent cleavage of tight-junction proteins at the blood-brain barrier (BBB) are regulated by microglia (da Fonseca et al., 2014; Kauppinen and Swanson, 2005; Rivera et al., 2002; Rosenberg et al., 2001; Shigemoto-Mogami et al., 2018) we next investigated MMP-9 activity in the striatum and cortex of both WT and STEP KO mice 18 h after reperfusion. FIGS. 16A-H show that ischemic stroke enhances MMP-9 activity, tight-junction protein degradation and BBB permeability in STEP KO mice. MMP-9 activity was measured by a FRET peptide immunoassay in striatum (FIG. 16A) and cortex (FIG. 16B) of WT and STEP KO mice 18 h after post-MCAO reperfusion. Data represents mean±SEM and expressed as relative fluorescent unit (RFU) value for each sample (n=8-9 mice/group). Degradation of tight junction proteins ZO-1 and occludin was evaluated by immunoblot analysis of striatal (FIGS. 16C, E) and cortical (FIGS. 16D, F) lysates obtained from ipsilateral hemisphere of WT and STEP KO mice, 24 h after post-MCAO reperfusion. Blots were analyzed with anti-ZO-1 antibody (FIGS. 16C, D) or anti-occludin antibody (FIGS. 16E, F) (upper panels) and then re-probed with anti-β-tubulin antibody (lower panels). Bar diagrams represents mean±SEM (n=8-12 mice/group). IgG concentration in the striatal (FIG. 16G) and cortical (FIG. 16H) lysates obtained from ipsilateral hemisphere of WT and STEP KO mice 24 h after post-MCAO reperfusion was measured by enzyme immunoassay. Values are represented as mean±SEM (n=7-10 mice/group). *p<0.05, **p<0.001 and ***p<0.001 from WT.

The results show a significant increase in MMP-9 activity in the ipsilateral striatum and cortex of STEP KO mice as compared to WT littermates (FIGS. 16A, B). Subsequent studies evaluated the endogenous levels of the scaffolding protein ZO-1 and the transmembrane phosphoprotein occludin, two tight junction proteins that are components of the BBB and substrates of MMP-9 (Asahi et al., 2001; Cummins, 2012). While ZO-1 is known to play a role in the regulation of tight junction protein complex through its interaction with occludin and actin cytoskeleton, the monomeric and oligomeric forms of occludin links adjacent endothelial cells to form the paracellular barrier (Cummins, 2012; Furuse et al., 1994; McCaffrey et al., 2007). Our findings show that 24 h after reperfusion in STEP KO mice ZO-1 level is significantly reduced in both the ipsilateral striatum and cortex as compared to the WT littermates (FIG. 16C, D). A significant reduction in the level of the dimeric form of occludin (˜125 kDa) is also observed in the ipsilateral striatum and cortex of STEP KO mice, when compared to WT littermates (FIG. 16E, F). Finally, to determine whether BBB permeability changes with loss of tight junction proteins we measured the levels of IgG in the ischemic brain from both WT and STEP KO mice 24 h after MCAO and reperfusion (Diamond et al., 2013). Our findings demonstrate that IgG level is significantly elevated in the ipsilateral striatum and cortex of STEP deficient mice, when compared to WT littermates (FIG. 16G, H), confirming increased BBB permeability in the STEP KO mice.

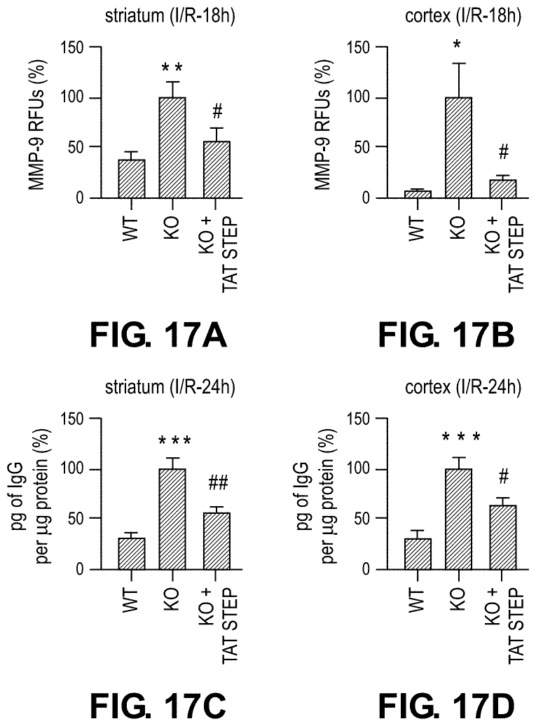

We next tested whether intravenous administration of the STEP peptide mimetic at the onset of reperfusion has any effect on MMP-9 activity and BBB permeability. For these experiments STEP KO mice were subjected to 30 min MCAO followed by intravenous administration of a single dose of vehicle or STEP-derived peptide (3 nmol/g) (FIG. 10) at the onset of reperfusion. They were then subjected to reperfusion for 18 or 24 hours. At 18 h of post-MCAO reperfusion, lysates from (A) striatum (FIG. 17A) and cortex (FIG. 17B) were processed for measurement of MMP-9 activity using FRET peptide-based immunoassay. At 24 h of post-MCAO reperfusion, lysates from striatum (FIG. 17C) and cortex (FIG. 17D) were processed for measurement of IgG concentration by enzyme immunoassay. Values of MMP-9 activity and IgG levels are represented as mean±SEM (7-10 mice/group). *p<0.05, **p<0.01 and ***p<0.0001 from WT; ^{#}p<0.05 and ^{##}p<0.001 from KO.

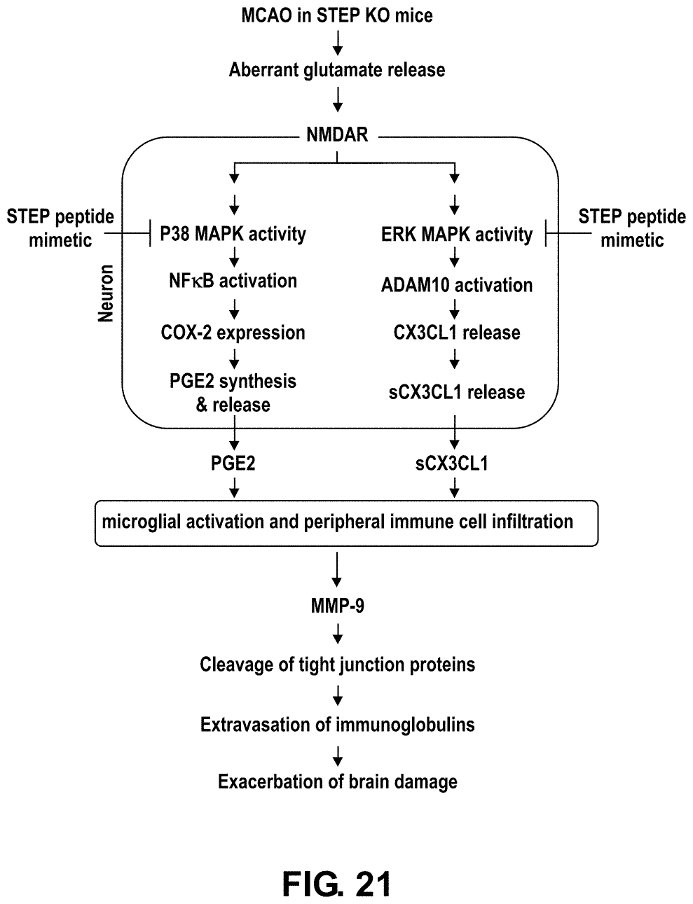

The extent of MMP-9 activity, analyzed 18 h after post-ischemic reperfusion, shows a significant decrease in both striatum and cortex of peptide treated mice (FIG. 17A, B). In addition, a significant reduction in extravasation of IgG is observed in the peptide treated mice 24 h after the onset of reperfusion (FIG. 17C, D). Together these findings confirm a role of STEP in regulating a signaling cascade (FIG. 21) that is involved in the potentiation of post-ischemic inflammatory responses in the brain.

Efficacy of STEP Peptide Mimetic in Regulating ERK MAPK Phosphorylation and Soluble CX3CL1 Release from Neurons Following an Excitotoxic Insult.

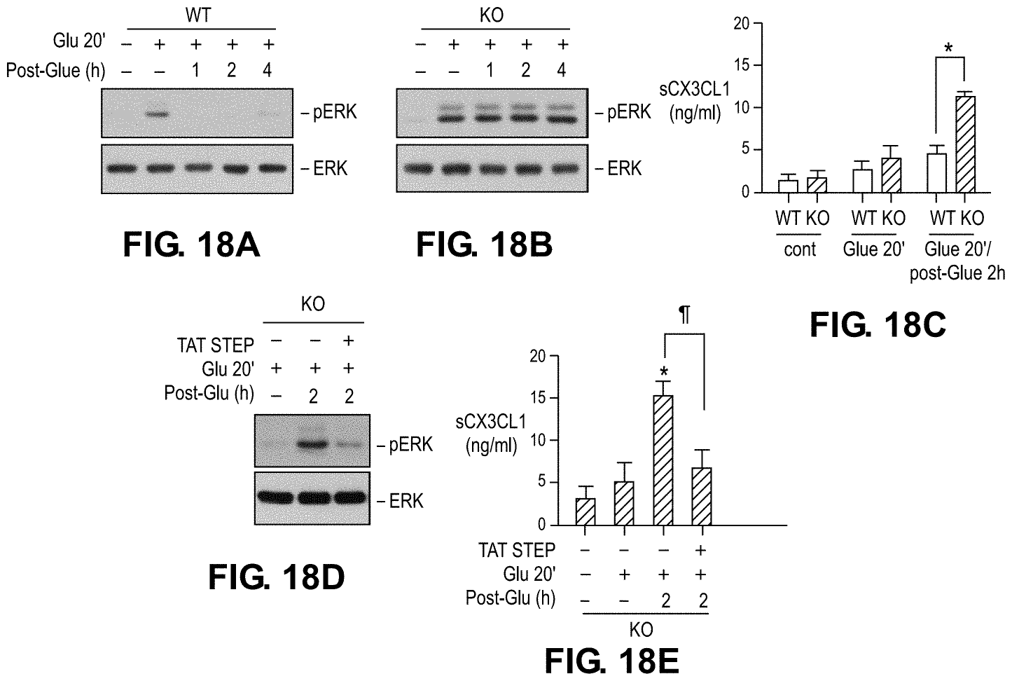

To further evaluate the effect of STEP gene deletion on glutamate-mediated ERK MAPK phosphorylation, in subsequent studies neuron cultures from WT (FIG. 18A) and STEP KO (FIG. 18B) mice were treated with 50 μM glutamate (Glu) for 20 min and then maintained in its original medium for the specified times (1 h, 2 h or 4 h). Equal amounts of protein from each sample was processed for immunoblot analysis using anti-phospho ERK MAPK (upper panel) and -ERK MAPK (lower panel) antibodies. Neuron cultures from WT and STEP KO mice were treated with 50 μM glutamate (Glu) for 20 min and then maintained in its original medium for 2 h. Equal amount of culture medium from each sample was analyzed for soluble CX3CL1 levels using ELISA. Values are expressed as mean±SEM. *, indicates significant difference between glutamate treated WT and STEP KO culture at post-glutamate time of 2 h (p<0.05). Neuron cultures from STEP KO mice were treated with 50 μM glutamate (Glu) in the absence or presence of TAT-STEP-myc peptide and then maintained in the original medium in the presence of the peptide for 2 h. Cell lysates with equal amount of protein from each sample was analyzed by immunoblotting using anti-phospho ERK MAPK (upper panel) and -ERK MAPK (lower panel) antibodies. Equal amount of culture medium from each sample was analyzed for soluble CX3CL1 levels using ELISA. Values are expressed as mean±SEM. *, indicates significant difference from untreated control culture (p<0.05). ^{¶}, indicates significant difference from glutamate treated STEP KO culture at post-glutamate time of 2 h (p<0.05).

In neuronal lysates from WT mice a transient increase in ERK MAPK phosphorylation is observed within 20 min of glutamate stimulation (FIG. 18A, upper panel). However, in neuronal lysates from STEP KO mice ERK MAPK phosphorylation remained sustained throughout the duration of the insult and recovery (FIG. 18B, upper panel). These findings indicate that of loss of endogenous STEP could lead to prolonged activation of ERK MAPK signaling pathway in neurons following an excitotoxic insult. In subsequent studies neuron cultures from WT and STEP KO mice were treated with 50 μM glutamate (20 min) followed by recovery for 2 h. Cell culture media were analyzed by ELISA to assess soluble CX3CL1 released following glutamate stimulation and recovery. The results show that glutamate treatment had no significant effect on soluble CX3CL1 release from WT mice neurons. However, soluble CX3CL1 release increased significantly from STEP KO mice neurons within 2 h of the recovery phase (FIG. 18C).

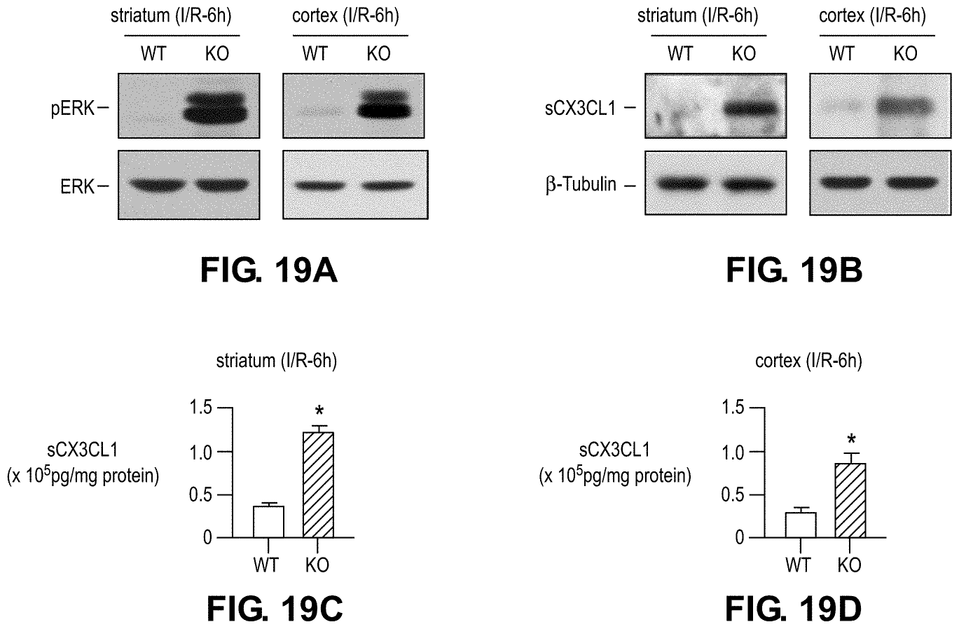

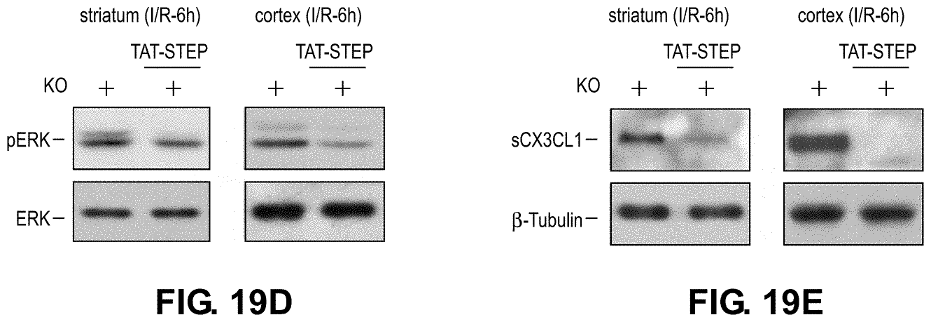

To test the hypothesis that the absence of endogenous STEP leads to upregulation ERK MAPK and soluble CX3CL1 release, neuron cultures from STEP KO mice were preincubated with the TAT-STEP-myc peptide (30 min) before exposure to glutamate (20 min), followed by recovery (2 h). WT and STEP KO mice were subjected to MCAO (30 min) followed by reperfusion for 6 h. Tissue extracts with equal amount of protein from the ipsilateral striatum and cortex was analyzed with anti-phospho-ERK MAPK (upper panels) and -ERK MAPK (lower panel) antibodies (FIG. 19A) or anti-CX3CL1 (upper panel) and anti-β-tubulin antibodies (lower panel) (FIG. 19B). CX3L1 level was measured by ELISA in the supernatants obtained from ipsilateral striatum and cortex of both WT and STEP KO mice. In FIG. 19C, the data are expressed as picograms (pg) of CX3CL1 level per milligram (mg) of protein. Bar diagram represent mean±SEM (n=3-4 mice/group). *, indicates significant difference between WT I/R 6 h and KO I/R 6 h (p<0.05). (D-E) STEP KO mice were subjected to MCAO (30 min) followed by reperfusion for 6 h. The STEP peptide mimetic (TAT-STEP-Myc) was administered in a subset of STEP KO mice at the onset of reperfusion. ERK MAPK phosphorylation (FIG. 19D) and CX3CL1 protein level (FIG. 19E) were evaluated by immunoblot analysis of tissue extracts from ipsilateral striatum and cortex (upper panels). Blots were re-probed with anti-ERK MAPK (FIG. 19D) or anti-β-tubulin antibody (FIG. 19E) (lower panels) Immunoblot analysis showed that STEP peptide mimetic blocked the phosphorylation of ERK MAPK assessed 2 h after a 20 min glutamate exposure (FIG. 19D). Evaluation of CX3CL1 level in the media 2 h after stimulation showed significant reduction in CX3CL1 release (FIG. 19E). These findings highlight the role of STEP in regulating an additional inflammatory response in neurons through ERK MAPK signaling, following an excitotoxic insult.

### Post-Ischemic Functional Deficit in STEP Deficient Mice

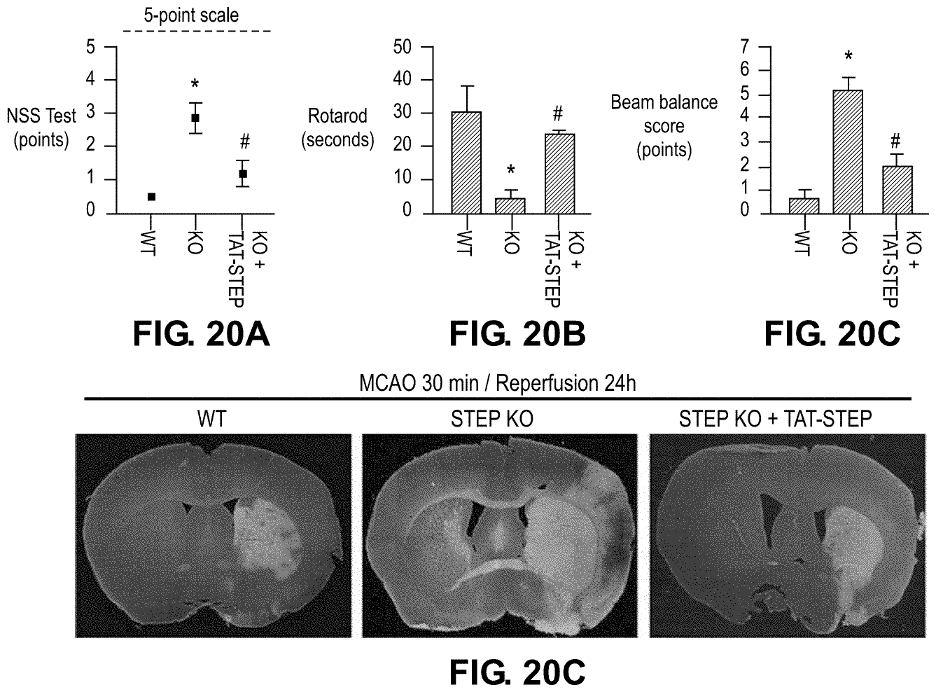

To determine whether the significant reduction of inflammatory response in STEP KO mice following treatment with the STEP peptide mimetic (FIG. 10) could also attenuate neurological and motor deficit, both WT and STEP KO mice were subjected to MCAO (30 min) followed by reperfusion for 24 h. A subset of ischemic STEP KO mice were treated with a single intravenous injection of the STEP peptide mimetic (3 nmol/g) at the onset of reperfusion. At 24 h after reperfusion WT mice shows minimal decline in neurological function as assessed by the modified neurological severity score. In contrast, the STEP KO mice shows a severe decline in neurological function (FIG. 20A). Motor function, as assessed by rotarod and beam balance tests also shows a significant decline in STEP KO mice, when compared to the WT littermates (FIG. 20B, C). Intervention with the STEP peptide mimetic shows significantly enhanced neurological and motor function in STEP KO mice, when compared to vehicle-treated STEP KO controls (FIG. 20A-C). Following functional studies, the mice were processed to evaluate the extent of brain damage using Fluoro-Jade staining. The representative photomicrograph shows that deletion of STEP gene leads to exacerbation of ischemic brain injury, while restoration of STEP signaling with intravenous administration of the STEP peptide mimetic reduces ischemic brain damage (FIG. 20D), which is consistent with our earlier finding (Deb et al., 2013). Together these findings confirm a role of STEP in regulating a signaling cascade that is involved in the potentiation of post-ischemic neuroinflammatory response in the brain (FIG. 21).

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

### REFERENCES

Lewerenz J, Maher P. (2015) Chronic Glutamate Toxicity in Neurodegenerative Diseases-What is the Evidence? Front Neurosci. 9:469.
Olney J W. (1986) Inciting excitotoxic cytocide among central neurons. Adv Exp Med Biol. 203:631-45.
Dong X X, Wang Y, Qin Z H. (2009) Molecular mechanisms of excitotoxicity and their relevance to pathogenesis of neurodegenerative diseases. Acta Pharmacol Sin. 30(4):379-87.
Olloquequi J, Cornejo-Cordova E, Verdaguer E, Soriano F X, Binvignat O, Auladell C, Camins A. (2018) Excitotoxicity in the pathogenesis of neurological and psychiatric disorders: Therapeutic implications. J Psychopharmacol. 32(3):265-275.
Lai T W, Zhang S, Wang Y T. (2014) Excitotoxicity and stroke: identifying novel targets for neuroprotection. Prog Neurobiol. 115:157-88.
Lodge D. (2009) The history of the pharmacology and cloning of ionotropic glutamate receptors and the development of idiosyncratic nomenclature. Neuropharmacology 56(1):6-21.
Spooren W, Lesage A, Lavreysen H, Gasparini F, Steckler T. (2010) Metabotropic glutamate receptors: their therapeutic potential in anxiety. Curr Top Behav Neurosci. 2:391-413.
Waxman E A, Lynch D R. (2005) N-methyl-D-aspartate receptor subtypes: multiple roles in excitotoxicity and neurological disease. Neuroscientist. 11(1):37-49.
Lynch D R, Guttmann R P. (2002) Excitotoxicity: perspectives based on N-methyl-D-aspartate receptor subtypes. J Pharmacol Exp Ther. 300(3):717-23.
Wang X, Michaelis E K. (2010) Selective neuronal vulnerability to oxidative stress in the brain. Front Aging Neurosci. 2:12.
Dantzer R, Walker A K. (2014) Is there a role for glutamate-mediated excitotoxicity in inflammation-induced depression? J Neural Transm (Vienna). 121(8):925-32.
Viviani B, Boraso M, Marchetti N, Marinovich M. (2014) Perspectives on neuroinflammation and excitotoxicity: a neurotoxic conspiracy? Neurotoxicology. 43:10-20.
Haroon E, Miller A H, Sanacora G. (2017) Inflammation, Glutamate, and Glia: A Trio of Trouble in Mood Disorders. Neuropsychopharmacology. 42(1):193-215.
Lombroso P J, Naegele J R, Sharma E, Lerner M. (1993) A protein tyrosine phosphatase expressed within dopaminoceptive neurons of the basal ganglia and related structures. J Neurosci. 13(7):3064-74.
Boulanger L M, Lombroso P J, Raghunathan A, During M J, Wahle P, Naegele J R. (1995) Cellular and molecular characterization of a brain-enriched protein tyrosine phosphatase. J Neurosci. 15(2):1532-44.
Paul S, Connor J A. (2010) NR2B-NMDA receptor-mediated increases in intracellular Ca2+ concentration regulate the tyrosine phosphatase, STEP, and ERK MAP kinase signaling. J Neurochem. 114(4):1107-18.
Paul S, Snyder G L, Yokakura H, Picciotto M R, Nairn A C, Lombroso P J. (2000) The Dopamine/D1 receptor mediates the phosphorylation and inactivation of the protein tyrosine phosphatase STEP via a PKA-dependent pathway. J Neurosci. 20(15):5630-8.
Paul S, Nairn A C, Wang P, Lombroso P J. (2003) NMDA-mediated activation of the tyrosine phosphatase STEP regulates the duration of ERK signaling. Nat Neurosci. 6(1):34-42.
Poddar R, Deb I, Mukherjee S, Paul S. (2010) NR2B-NMDA receptor mediated modulation of the tyrosine phosphatase STEP regulates glutamate induced neuronal cell death. J Neurochem. 115(6):1350-62.
Deb I, Manhas N, Poddar R, Rajagopal S, Allan A M, Lombroso P J, Rosenberg G A, Candelario-Jalil E, Paul S. (2013) Neuroprotective role of a brain-enriched tyrosine phosphatase, STEP, in focal cerebral ischemia. J Neurosci. 33(45):17814-26.
Wu Q J, Tymianski M. (2018) Targeting NMDA receptors in stroke: new hope in neuroprotection. Mol Brain. 11(1):15.
Rajagopal S, Deb I, Poddar R, Paul S. (2016) Aging is associated with dimerization and inactivation of the brain-enriched tyrosine phosphatase STEP. Neurobiol Aging. 41:25-38.
Dong P, Zhao J, Zhang Y, Dong J, Zhang L, Li D, Li L, Zhang X, Yang B, Lei W. (2014) Aging causes exacerbated ischemic brain injury and failure of sevoflurane post-conditioning: role of B-cell lymphoma-2. Neuroscience. 275:2-11.
Hwang D, Jang B C, Yu G, Boudreau M. (1997) Expression of mitogen-inducible cyclooxygenase induced by lipopolysaccharide: mediation through both mitogen-activated protein kinase and NF-kappaB signaling pathways in macrophages. Biochem Pharmacol. 54(1):87-96.
Ridley S H, Dean J L, Sarsfield S J, Brook M, Clark A R, Saklatvala J. (1998) A p38 MAP kinase inhibitor regulates stability of interleukin-1-induced cyclooxygenase-2 mRNA. FEB Lett. 439(1-2):75-80.
Paul A, Cuenda A, Bryant C E, Murray J, Chilvers E R, Cohen P, Gould G W, Plevin R. (1999) Involvement of mitogen-activated protein kinase homologues in the regulation of lipopolysaccharide-mediated induction of cyclo-oxygenase-2 but not nitric oxide synthase in RAW 264.7 macrophages. Cell Signal. 11(7):491-7.
Sheridan G K, Murphy K J. (2013) Neuron-glia crosstalk in health and disease: fractalkine and CX3CR1 take center stage. Open Biol. 3(12):130181.
Nishiyori A, Minami M, Ohtani Y, Takami S, Yamamoto J, Kawaguchi N, Kume T, Akaike A, Satoh M. (1998) Localization of fractalkine and CX3CR1 mRNAs in rat brain: does fractalkine play a role in signaling from neuron to microglia? FEBS Lett. 429(2):167-72.
Rostene W, Kitabgi P, Parsadaniantz S M. (2007) Chemokines: a new class of neuromodulator? Nat Rev Neurosci. 8(11):895-903.
Harrison J K, Jiang Y, Chen S, Xia Y, Maciejewski D, McNamara R K, Streit W J, Salafranca M N, Adhikari S, Thompson D A, Botti P, Bacon K B, Feng L. (1998) Role for neuronally derived fractalkine in mediating interactions between neurons and CX3CR1-expressing microglia. Proc Natl Acad Sci USA. 95(18):10896-901.
Wan X Z, Li B, Li Y C, Yang X L, Zhang W, Zhong L, Tang S J. (2012) Activation of NMDA receptors upregulates a disintegrin and metalloproteinase 10 via a Wnt/MAPK signaling pathway. J Neurosci. 32(11):3910-6.
Hundhausen C, Misztela D, Berkhout T A, Broadway N, Saftig P, Reiss K, Hartmann D, Fahrenholz F, Postina R, Matthews V, Kallen K J, Rose-John S, Ludwig A. (2003) The disintegrin-like metalloproteinase ADAM10 is involved in constitutive cleavage of CX3CL1 (fractalkine) and regulates CX3CL1-mediated cell-cell adhesion. Blood. 102(4):1186-95.
Hundhausen C, Schulte A, Schulz B, Andrzejewski M G, Schwarz N, von Hundelshausen P, Winter U, Paliga K, Reiss K, Saftig P, Weber C, Ludwig A. (2007) Regulated shedding of transmembrane chemokines by the disintegrin and metalloproteinase 10 facilitates detachment of adherent leukocytes. J Immunol. 178(12):8064-72.
Rajagopal S, Yang C, DeMars K M, Poddar R, Candelario-Jalil E, Paul S. (2021) Regulation of post-ischemic inflammatory response: A novel function of the neuronal tyrosine phosphatase STEP. Brain Behav Immun January 8: S0889-1591(21) 00003-9. doi: 10.1016/j.bbi.2020.12.034. Epub ahead of print. PMID: 33422638.
Ainslie P N, Cotter J D, George K P, Lucas S, Murrell C, Shave R, Thomas K N, Williams M J, Atkinson G. (2008) Elevation in cerebral blood flow velocity with aerobic fitness throughout healthy human ageing. J Physiol. 586(16):4005-10.
De Vis J B, Hendrikse J, Bhogal A, Adams A, Kappelle U, Petersen E T. (2015) Age-related changes in brain hemodynamics; A calibrated MRI study. Hum Brain Mapp. 36(10):3973-87.
Candelario-Jalil E, Paul S. (2020) Impact of aging and comorbidities on ischemic stroke outcomes in preclinical animal models: A translational perspective. Exp Neurol. 335:113494.
Macri M A, D'Alessandro N, Di Giulio C, Di Iorio P, Di Luzio S, Giuliani P, Esposito E, Pokorski M. (2010) Region-specific effects on brain metabolites of hypoxia and hyperoxia overlaid on cerebral ischemia in young and old rats: a quantitative proton magnetic resonance spectroscopy study. J Biomed Sci. 17(1):14.
Østergaard L, Engedal T S, Moreton F, Hansen M B, Wardlaw J M, Dalkara T, Markus H S, Muir K W. (2016) Cerebral small vessel disease: Capillary pathways to stroke and cognitive decline. J Cereb Blood Flow Metab. 36(2):302-25.
Hekimi S, Lapointe J, Wen Y. (2011) Taking a “good” look at free radicals in the aging process. Trends Cell Biol. 21(10):569-76.
Yankner B A, Lu T, Loerch P. (2008) The aging brain. Annu Rev Pathol. 3:41-66.
Currais A, Maher P. (2013) Functional consequences of age-dependent changes in glutathione status in the brain. Antioxid Redox Signal. 19(8):813-22.
Ballatori N, Krance S M, Notenboom S, Shi S, Tieu K, Hammond C L. (2009) Glutathione dysregulation and the etiology and progression of human diseases. Biol Chem.
Emir U E, Raatz S, McPherson S, Hodges J S, Torkelson C, Tawfik P, White T, Terpstra M. (2011) Noninvasive quantification of ascorbate and glutathione concentration in the elderly human brain. NMR Biomed. 24(7):888-94.
Deb I, Poddar R, Paul S. (2011) Oxidative stress-induced oligomerization inhibits the activity of the non-receptor tyrosine phosphatase STEP61. J Neurochem. 116(6):1097-111.
Munoz L, Ammit A J. (2010) Targeting p38 MAPK pathway for the treatment of Alzheimer's disease. Neuropharmacology. 58(3):561-8.
Kim, E. K., Choi, E J. (2015) Compromised MAPK signaling in human diseases: an update. Arch. Toxicol. 89: 867-882.
He J, Zhong W, Zhang M, Zhang R, Hu W. (2018) P38 Mitogen-activated Protein Kinase and Parkinson's Disease. Transl Neurosci. 9:147-153.
Lin L L, Wartmann M, Lin A Y, Knopf J L, Seth A, Davis R J. (1993) cPLA2 is phosphorylated and activated by MAP kinase. Cell. 72(2):269-78.
Kramer R M, Roberts E F, Um S L, Borsch-Haubold A G, Watson S P, Fisher M J, Jakubowski J A. (1996) p38 mitogen-activated protein kinase phosphorylates cytosolic phospholipase A2 (cPLA2) in thrombin-stimulated platelets. Evidence that proline-directed phosphorylation is not required for mobilization of arachidonic acid by cPLA2. J Biol Chem. 271 (44):27723-9.
Lasa, M., K. R. Mahtani, A. Finch, G. Brewer, J. Saklatvala, and A. R. Clark. (2000) Regulation of cyclooxygenase 2 mRNA stability by the mitogen-activated protein kinase p38 signaling cascade. Mol Cell Biol. 20:4265-4274.
Svensson, C. I., X. Y. Hua, A. A. Protter, H. C. Powell, and T. L. Yaksh. (2003) Spinal p38 MAP kinase is necessary for NMDA-induced spinal PGE(2) release and thermal hyperalgesia. Neuroreport 14:1153-1157.
Poddar R, Rajagopal S, Winter L, Allan A M, Paul S. (2019) A peptide mimetic of tyrosine phosphatase STEP as a potential therapeutic agent for treatment of cerebral ischemic stroke. J Cereb Blood Flow Metab. 39(6):1069-1084.
Graeber M B, Li W, Rodriguez M L. (2011) Role of microglia in CNS inflammation. FEBS Lett. 585(23):3798-805.
Prinz M, Priller J. (2017) The role of peripheral immune cells in the CNS in steady state and disease. Nat Neurosci. 20(2):136-144.
Deep S N, Mitra S, Rajagopal S, Paul S, Poddar R. (2019) GluN2A-NMDA receptor-mediated sustained Ca2+ influx leads to homocysteine-induced neuronal cell death. J Biol Chem. 294(29):11154-11165.
Laemmli U K (1970) Cleavage of structural proteins during the assembly of the head of bacteriophage T4. Nature 227:680-685.
Swanson R A, Morton M T, Tsao-Wu G, Savalos R A, Davidson C, Sharp F R. (1990) A semiautomated method for measuring brain infarct volume. J Cereb Blood Flow Metab. 10(2):290-3.
Minghetti L. (2004) Cyclooxygenase-2 (COX-2) in inflammatory and degenerative brain diseases. J Neuropathol Exp Neurol. 63(9):901-10.
Strauss K I, Marini A M. (2002) Cyclooxygenase-2 inhibition protects cultured cerebellar granule neurons from glutamate-mediated cell death. J Neurotrauma. 19(5):627-38.
Shen Y, Kishimoto K, Linden D J, Sapirstein A. (2007) Cytosolic phospholipase A(2) alpha mediates electrophysiologic responses of hippocampal pyramidal neurons to neurotoxic NMDA treatment. Proc Natl Acad Sci USA. 104(14):6078-83.
Shelat P B, Chalimoniuk M, Wang J H, Strosznajder J B, Lee J C, Sun A Y, Simonyi A, Sun G Y. (2008) Amyloid beta peptide and NMDA induce ROS from NADPH oxidase and AA release from cytosolic phospholipase A2 in cortical neurons. J Neurochem. 106(1):45-55.
Yamamoto K, Arakawa T, Ueda N, Yamamoto S. (1995) Transcriptional roles of nuclear factor kappa B and nuclear factor-interleukin-6 in the tumor necrosis factor alpha-dependent induction of cyclooxygenase-2 in MC3T3-E1 cells. J Biol Chem. 270:31315-31320.
Kaltschmidt B, Linker R A, Deng J, Kaltschmidt C. (2002) Cyclooxygenase-2 is a neuronal target gene of NF-kappaB. BMC Mol Biol. 3:16.
Ackerman W E 4th, Summerfield T L, Vandre D D, Robinson J M, Kniss D A. (2008) Nuclear factor kappa B regulates inducible prostaglandin E synthase expression in human amnion mesenchymal cells. Biol Reprod. 78:68-76.
Guo R M, Xu W M, Lin J C, Mo L Q, Hua X X, Chen P X, Wu K, Zheng D D, Feng J Q. (2013) Activation of the p38 MAPK/NF-κB pathway contributes to doxorubicin-induced inflammation and cytotoxicity in H9c2 cardiac cells. Mol Med Rep. 8(2):603-8.
Shi G, Li D, Fu J, Sun Y, Li Y, Qu R, Jin X, Li D. (2015) Upregulation of cyclooxygenase-2 is associated with activation of the alternative nuclear factor kappa B signaling pathway in colonic adenocarcinoma. Am J Transl Res. 7(9):1612-20.
Kaltschmidt B, Widera D, Kaltschmidt C. (2005) Signaling via NF-kappaB in the nervous system. Biochim Biophys Acta. 1745(3):287-99.
Karin M, Ben-Neriah Y. (2000) Phosphorylation meets ubiquitination: the control of NF-[kappa]B activity. Annu Rev Immunol. 18:621-63.
Barone, F. C., Irving, E. A., Ray, A. M., Lee, J. C., Kassis, S., Kumar, S., Badger, A. M., Legos, J. J., Erhardt, J. A., Ohlstein, E. H., et al. (2001a). Inhibition of p38 mitogen-activated protein kinase provides neuroprotection in cerebral focal ischemia. Medicinal research reviews. 21: 129-145.
Barone, F. C., Irving, E. A., Ray, A. M., Lee, J. C., Kassis, S., Kumar, S., Badger, A. M., White, R. F., McVey, M. J., Legos, J. J., et al. (2001b). SB 239063, a second-generation p38 mitogen-activated protein kinase inhibitor, reduces brain injury and neurological deficits in cerebral focal ischemia. The Journal of pharmacology and experimental therapeutics. 296: 312-321.
Nozaki, K., Nishimura, M., and Hashimoto, N. (2001). Mitogen-activated protein kinases and cerebral ischemia. Mol Neurobiol. 23: 1-19.
Miettinen, S., Fusco, F. R., Yrjanheikki, J., Keinanen, R., Hirvonen, T., Roivainen, R., Narhi, M., Hokfelt, T., and Koistinaho, J. (1997). Spreading depression and focal brain ischemia induce cyclooxygenase-2 in cortical neurons through N-methyl-D-aspartic acid-receptors and phospholipase A2. Proceedings of the National Academy of Sciences of the United States of America. 94: 6500-6505.
Nito, C., Kamada, H., Endo, H., Niizuma, K., Myer, D. J., and Chan, P. H. (2008). Role of the p38 mitogen-activated protein kinase/cytosolic phospholipase A2 signaling pathway in blood-brain barrier disruption after focal cerebral ischemia and reperfusion. J Cereb Blood Flow Metab. 28: 1686-1696.
Nogawa, S., Zhang, F., Ross, M. E., and Iadecola, C. (1997). Cyclo-oxygenase-2 gene expression in neurons contributes to ischemic brain damage. J Neurosci. 17: 2746-2755.
Sasaki, T., Kitagawa, K., Yamagata, K., Takemiya, T., Tanaka, S., Omura-Matsuoka, E., Sugiura, S., Matsumoto, M., and Hori, M. (2004). Amelioration of hippocampal neuronal damage after transient forebrain ischemia in cyclooxygenase-2-deficient mice. J Cereb Blood Flow Metab. 24: 107-113.
Smith, W. L., DeWitt, D. L., and Garavito, R. M. (2000). Cyclooxygenases: structural, cellular, and molecular biology. Annual review of biochemistry. 69: 145-182.
del Zoppo, G. J., Milner, R., Mabuchi, T., Hung, S., Wang, X., Berg, G. I., and Koziol, J. A. (2007). Microglial activation and matrix protease generation during focal cerebral ischemia. Stroke 38, 646-651.
Kreutzberg, G. W. (1996). Microglia: a sensor for pathological events in the CNS. Trends Neurosci. 19: 312-318.
Mabuchi, T., Kitagawa, K., Ohtsuki, T., Kuwabara, K., Yagita, Y., Yanagihara, T., Hori, M., and Matsumoto, M. (2000). Contribution of microglia/macrophages to expansion of infarction and response of oligodendrocytes after focal cerebral ischemia in rats. Stroke. 31:
Wang, Q., Tang, X. N., and Yenari, M. A. (2007). The inflammatory response in stroke. J Neuroimmunol. 184: 53-68.
Ito, D., Tanaka, K., Suzuki, S., Dembo, T., and Fukuuchi, Y. (2001) Enhanced expression of Iba1, ionized calcium-binding adapter molecule 1, after transient focal cerebral ischemia in rat brain. Stroke. 32: 1208-1215.
Zhang, Z., Chopp, M., and Powers, C. (1997). Temporal profile of microglial response following transient (2 h) middle cerebral artery occlusion. Brain Res. 744: 189-198.
da Fonseca, A. C., Matias, D., Garcia, C., Amaral, R., Geraldo, L. H., Freitas, C., and Lima, F. R. (2014). The impact of microglial activation on blood-brain barrier in brain diseases. Front Cell Neurosci. 8: 362.
Kauppinen, T. M., and Swanson, R. A. (2005). Poly(ADP-ribose) polymerase-1 promotes microglial activation, proliferation, and matrix metalloproteinase-9-mediated neuron death. J Immunol. 174: 2288-2296.
Rivera, S., Ogier, C., Jourquin, J., Timsit, S., Szklarczyk, A. W., Miller, K., Gearing, A. J., Kaczmarek, L., and Khrestchatisky, M. (2002). Gelatinase B and TIMP-1 are regulated in a cell- and time-dependent manner in association with neuronal death and glial reactivity after global forebrain ischemia. Eur J Neurosci. 15: 19-32.
Rosenberg, G. A., Cunningham, L. A., Wallace, J., Alexander, S., Estrada, E. Y., Grossetete, M., Razhagi, A., Miller, K., and Gearing, A. (2001) Immunohistochemistry of matrix metalloproteinases in reperfusion injury to rat brain: activation of MMP-9 linked to stromelysin-1 and microglia in cell cultures. Brain Res. 893: 104-112.
Shigemoto-Mogami, Y., Hoshikawa, K., and Sato, K. (2018). Activated Microglia Disrupt the Blood-Brain Barrier and Induce Chemokines and Cytokines in a Rat in vitro Model. Front Cell Neurosci. 12: 494.
Asahi, M., Wang, X., Mori, T., Sumii, T., Jung, J. C., Moskowitz, M. A., Fini, M. E., and Lo, E. H. (2001). Effects of matrix metalloproteinase-9 gene knock-out on the proteolysis of blood-brain barrier and white matter components after cerebral ischemia. J Neurosci. 21: 7724-7732.
Cummins, P. M. (2012). Occludin: one protein, many forms. Mol Cell Biol. 32: 242-250.
Furuse, M., Itoh, M., Hirase, T., Nagafuchi, A., Yonemura, S., Tsukita, S., and Tsukita, S. (1994). Direct association of occludin with ZO-1 and its possible involvement in the localization of occludin at tight junctions. J Cell Biol. 127: 1617-1626.
McCaffrey, G., Staatz, W. D., Quigley, C. A., Nametz, N., Seelbach, M. J., Campos, C. R., Brooks, T. A., Egleton, R. D., and Davis, T. P. (2007). Tight junctions contain oligomeric protein asSDbly critical for maintaining blood-brain barrier integrity in vivo. J Neurochem. 103: 2540-2555.
Diamond, B., Honig, G., Mader, S., Brimberg, L., and Volpe, B. T. (2013). Brain-reactive antibodies and disease. Annu Rev Immunol. 31: 345-385.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for the prevention, treatment, or amelioration of a medical disease or condition associated with inflammation caused by glutamate excitotoxicity comprising administering to a patient suffering from the disease or condition, a peptide that constitutively binds at least one of p38 MAPK and ERK MAPK.

2. The method of claim 1 wherein the peptide is a synthetic peptide mimetic of at least a portion of a modified STEP protein.

3. The method of claim 2 wherein the synthetic peptide mimetic comprises at least a portion of the KIM domain of the STEP protein.

4. The method of claim 3 wherein the portion of the KIM domain in the synthetic peptide mimetic is modified to render the peptide constitutively active.

5. The method of claim 3 wherein the synthetic peptide mimetic comprises at least a portion of the KIM domain of the STEP protein.

6. The method of claim 5 wherein the portion of the KIS domain is modified to render the peptide resistant to degradation.

7. The method of claim 1 wherein the peptide comprises a sequence that renders the peptide cell-permeable.

8. A method for the prevention, treatment, or amelioration of a medical disease or condition associated with inflammation caused by glutamate excitotoxicity comprising administering to a patient suffering from the disease or condition, a peptide that interferes with at least one of the P38 MAPK-COX2-PGE₂ and ERK MAPK-CX3CL1-xCX3CL1 pathways.

9. The method of claim 8 wherein the peptide is a synthetic peptide mimetic of at least a portion of a modified STEP protein.

10. The method of claim 9 wherein the synthetic peptide mimetic comprises at least a portion of the KIM domain of the STEP protein.

11. The method of claim 10 wherein the portion of the KIM domain in the synthetic peptide mimetic is modified to render the peptide constitutively active.

12. The method of claim 10 wherein the synthetic peptide mimetic comprises at least a portion of the KIM domain of the STEP protein.

13. The method of claim 12 wherein the portion of the KIS domain is modified to render the peptide resistant to degradation.

14. The method of claim 8 wherein the peptide comprises a sequence that renders the peptide cell-permeable.
